(19) United States
(12) Patent Application Publication
(10) Pub. No.: US 2026/0294874 A1
(43) Pub. Date: Oct. 1, 2026

(54) COMPOSITIONS COMPRISING MODULATORS OF RIBONUCLEOPROTEIN GRANULE FORMATION AND DISSOLUTION AND METHODS OF USE THEREOF

(71) Applicant: University of Virginia Patent Foundation, Charlottesville, VA (US)

(72) Inventors: Ku-Lung Hsu, Cedar Park, TX (US); Anthony Michael Ciancone, Bethesda, MD (US)

(73) Assignee: University of Virginia Patent Foundation, Charlottesville, VA (US)

(21) Appl. No.: 19/475,385

(22) PCT Filed: Apr. 12, 2024
(86) PCT No.: PCT/US2024/024445

§ 371 Date: Oct. 14, 2025

Related U.S. Application Data
(60) Provisional application No. 63/458,909, filed on Apr. 12, 2023.

(51) Int. Cl. A61K 31/4196; A61K 31/4192

(52) U.S. Cl. CPC A61K 31/4196; A61K 31/4192

(57) ABSTRACT

Provided are sulfonyl triazole compounds as well as compositions thereof and methods for using the same for modulating the formation of ribonucleoprotein granules (RNPGs), treating symptoms of diseases or infections in subjects including but not limited to viral infections, cancers and/or tumors, and/or neurological diseases. In some embodiments, the sulfonyl triazole compounds have the structure of Formula (I) as defined herein.

### CROSS-REFERENCE TO RELATED APPLICATION

This application claims the benefit of U.S. Provisional Patent Application Ser. No. 63/458,909, filed Apr. 12, 2023, the disclosure of which is incorporated herein by reference in its entirety.

### GOVERNMENT SUPPORT

This invention was made with government support under CA044579, DA043571, GM132189, and GM144472 awarded by the National Institutes of Health. The government has certain rights in the invention.

### REFERENCE TO SEQUENCE LISTING XML

The Sequence Listing XML associated with the instant disclosure has been electronically submitted to the United States Patent and Trademark Office via the Patent Center as a 3,605 byte UTF-8-encoded XML file created on Apr. 12, 2024 and entitled “3062_199_PCT.xml”. The Sequence Listing submitted via Patent Center is hereby incorporated by reference in its entirety.

### TECHNICAL FIELD

The presently disclosed subject matter relates in some embodiments to methods and compositions for modulating the formation of ribonucleoprotein granules (RNPGs). In some embodiments, the presently disclosed subject matter relates to compositions and methods for treating symptoms of diseases and/or infections, particularly cancers, neurological diseases, and viral infections, with sulfonyl triazole compounds.

### BACKGROUND

Cellular RNA and protein accumulate in membraneless subcellular compartments referred to collectively as biomolecular condensates (Banani et al., 2017). Condensate formation is proposed to involve liquid-liquid phase separation (LLPS) of proteins and nucleic acids, can occur in response to cellular stimuli, and is associated with the regulation of RNA metabolism, translation, and signal transduction (Banani et al., 2017; Ivanov et al., 2019; Shin & Brangwynne, 2017). Stress granules (SGs) and processing bodies (PBs) are widely studied cytoplasmic ribonucleoprotein (RNP) granules that are implicated in post-transcriptional control of gene expression and cellular fitness although their specific functions remain to be fully elucidated (Ivanov et al., 2019).

SG and PB formation are driven by key granule-forming RNA-binding proteins (RBPs). These RNPs can self-organize into granule structures through protein-RNA, protein-protein and RNA-RNA interactions to mediate multiphase condensation (Collier & Schlesinger, 1986; Nover et al., 1989; Sheth & Parker, 2003; Kedersha & Anderson, 2007; Ivanov et al., 2019; Sanders et al., 2020; Yang et al., 2020). Cells form SGs under stress conditions while PBs exist constitutively but can increase in size and number with stress (Luo et al., 2018; Standart & Weil, 2018; Ivanov et al., 2019). SG formation is triggered by the integrated stress response through activation of kinases that phosphorylate eIF2a, resulting in translational arrest and accumulation of untranslated mRNA that promote protein and RNA condensation (Protter & Parker, 2016; Ivanov et al., 2019; Riggs et al., 2020). The SG-associated proteome includes proteins involved in RNA metabolism, mRNA translation, and essential SG-nucleating RBPs such as but not limited to Ras GTPase-activating protein-binding protein or G3BP (Decker & Parker, 2012; Stoecklin & Kedersha, 2013; Ivanov et al., 2019; Guillen-Boixet et al., 2020; Sanders et al., 2020; Yang et al., 2020). PBs are enriched for proteins involved in mRNA decay including mRNA-decapping enzymes (DCP1A and DCP2) and enhancer of mRNA-decapping proteins (EDC3 and EDC4; Brengues et al., 2005; Hubstenberger et al., 2017; Lavalee et al., 2021).

Aberrant regulation of RNP granules has been associated with an increasing number of disease states (Anderson et al., 2015; Reineke & Lloyd, 2015; Mackenzie et al., 2017; Baradaran-Heravi et al., 2020; Gordon et al., 2020; Hans et al., 2020; Marmor-Kollet et al., 2020; Cui et al., 2023). Cancer resistance to radiation or chemotherapeutics has been linked to formation of pro-survival SGs (Kwon et al., 2007; Arimoto et al., 2008; Fournier et al., 2010; Fujimura et al., 2012; Kaehler et al., 2014). PBs are reported to be regulated by alpha-synuclein, an aggregating protein genetically linked to Parkinson's disease (Hallacli et al., 2022). More generally, the ability to pharmacologically modulate disease-relevant condensates may provide new therapeutic opportunities but has so far proven challenging to identify compounds that directly engage granule forming proteins or RNA (Mitrea et al., 2022). One of the barriers is the difficulty in targeting the RNA binding interface of granule forming RBPs using small molecules because of the large, buried surface area and prevalence of positively charged amino acids in RNA-binding domains (RBDs; Kruger et al., 2018). RBPs also contain intrinsically disordered regions (IDRs) involved in RNA binding that have been historically difficult to target with small molecules (Hentze et al., 2018).

Pharmacological modulation of RNP granules has been primarily achieved through perturbation of major upstream biological processes leading to condensation. As such, these compounds affect multiple cellular pathways and are often unsuitable as specific modulators of RNP condensates due to off-target effects and cytotoxicity. General inhibitors of translational elongation (e.g., emetine and cycloheximide) have been used to disassemble SGs and stress-induced PBs (Cougot et al., 2004; Kedersha & Anderson, 2007; Colombrita et al., 2009). Toxins including pateamine A and hippuristanol can induce SGs in an eIF2a-independent manner by inactivating the RNA helicase eIF4A (Bordeleau et al., 2005; Low et al., 2005; Bordeleau et al., 2006; Kedersha & Anderson, 2007). Chemotherapy can trigger PB (Aizer et al., 2008; Ayache et al., 2015) and SG formation (Fournier et al., 2010; Kaehler et al., 2014; Anderson et al., 2015; Lavalee et al., 2021). High-throughput screening (HTS) has been pursued and while promising, the mode of action for these condensate-modulating compounds remains ill-defined (Sreedharan et al., 2008; Fang et al., 2019). Compounds that target SG proteins have been reported but direct binding remains to be demonstrated (Cai et al., 2021). Thus, apart from approved drugs with condensate modifying activity discovered after the fact (Patel et al., 2022), RNP granule modulators consist largely of toxic compounds or lead hits from HTS that lack known direct binding targets.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter relates to methods for modulating the formation of ribonucleoprotein granules (RNPGs). In some embodiments, the methods comprise, consist essentially of, or consist of contacting a sample comprising a protein with a sulfonyl triazole compound, thereby covalently modifying the protein. In some embodiments, the RNPG is a stress granule (SG) or a processing body (PB) and/or wherein the sulfonyl triazole compound is capable of covalently modifying an RNA granule induction-sensitive lysine and tyrosine site (RISKY) in the protein. In some embodiments, the sulfonyl triazole compound has a structure of Formula (I):

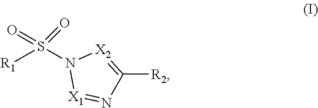

wherein X₁ and X₂ are each CH or N, subject to the proviso that when X₁ is CH, X₂ is N and when X₁ is N, X₂ is CH; R₁ is selected from alkyl, cycloalkyl, aralkyl, aryl, and substituted aryl; and R₂ is selected from alkyl, substituted alkyl, aralkyl, substituted aralkyl, aryl, and substituted aryl. In some embodiments, X₁ is N and X₂ is CH.

In some embodiments, R₁ is selected from the group consisting of isopropyl, cyclopropyl, naphthyl, pyridyl, phenyl, and substituted phenyl. In some embodiments, R₁ is substituted phenyl, wherein said substituted phenyl is phenyl substituted with one or more substituents selected from the group consisting of cyano, halo, alkyl, alkoxy, perfluoroalkyl, perfluoroalkoxy, phenyl, and —NH(═O)-alkyl, optionally wherein substituted phenyl is phenyl substituted with one or more substituents selected from the group consisting of cyano, bromo, fluoro, methyl, isopropyl, methoxy, trifluoromethyl, trifluoromethoxy, phenyl, and —NH(═O)-methyl.

In some embodiments, R₂ is selected from the group consisting of alkyl, sulfonyl-substituted alkyl, cycloalkyl, phenyl, pyridyl, and substituted phenyl. In some embodiments, R₂ is substituted phenyl, wherein said substituted phenyl is phenyl substituted with one or more substituents selected from the group consisting of halo, alkyl, perfluoroalkyl, alkoxy, and phenyl, optionally, fluoro, bromo, chloro, butyl, pentyl, trifluoromethyl, methoxy, and phenyl. In some embodiments, R₂ is selected from the group consisting of heptyl, hexyl, cyclohexyl, —CH₂—S(═O)₂—CH₃, pyridyl, and phenyl.

In some embodiments of the presently claimed methods, the modulating inhibits the formation of PB and/or SG, optionally wherein the compound is selected from the group consisting of:

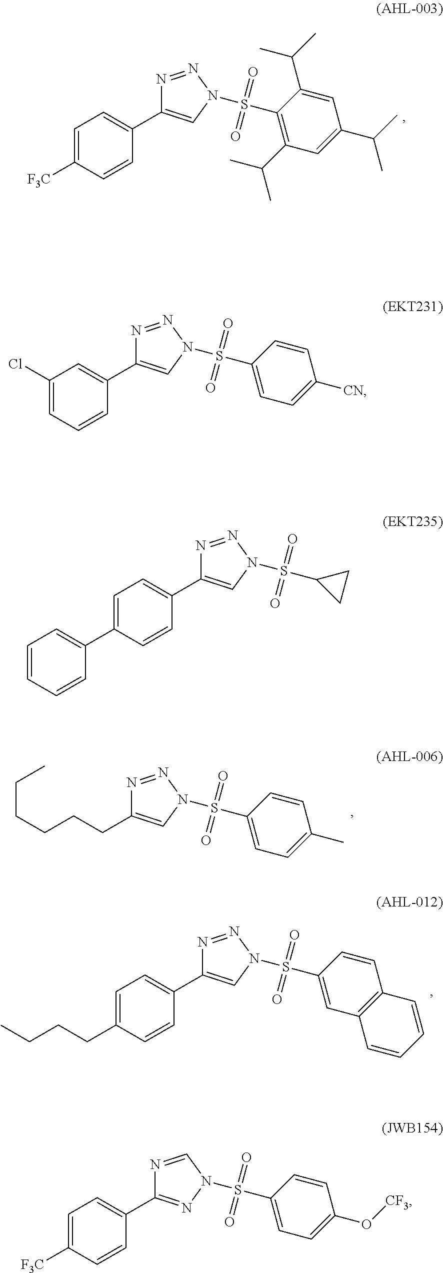

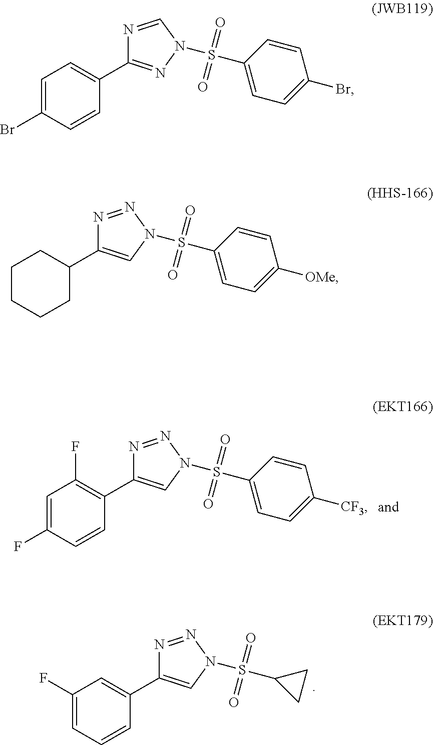 [SMILES: O=S(=O)(c1ccc(Br)cc1)n1cnc(-c2ccc(Br)cc2)n1 ; COc1ccc(S(=O)(=O)n2cc(C3CCCCC3)nn2)cc1 ; O=S(=O)(c1ccc(C(F)(F)F)cc1)n1cc(-c2ccc(F)cc2F)nn1 ; O=S(=O)(C1CC1)n1cc(-c2cccc(F)c2)nn1]

In some embodiments, the modulation induces the formation of PB and/or SG, optionally wherein the compound is selected from the group consisting of:

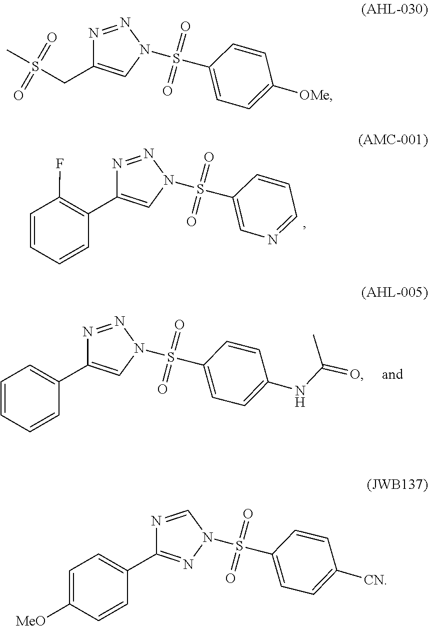 [SMILES: COc1ccc(S(=O)(=O)n2cc(CS(C)(=O)=O)nn2)cc1 ; O=S(=O)(c1cccnc1)n1cc(-c2ccccc2F)nn1 ; CC(=O)Nc1ccc(S(=O)(=O)n2cc(-c3ccccc3)nn2)cc1 ; "and" ; COc1ccc(-c2ncn(S(=O)(=O)c3ccc(C#N)cc3)n2)cc1]

In some embodiments, the protein is anti-RAS GTPase-activating protein-binding protein 1 (G3BP1), Parkinson diseases protein 1 (PARK7), or enhancer of mRNA-decapping protein 3 (EDC3).

In some embodiments, modulating the formation of RNPGs comprises inhibiting the multimerization of G3BP1, optionally wherein the method comprises contacting a sample comprising G3BP1 with

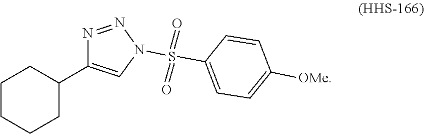 [SMILES: COc1ccc(S(=O)(=O)n2cc(C3CCCCC3)nn2)cc1]

In some embodiments, modulating the formation of RNPGs comprises inhibiting the multimerization of PARK7, optionally wherein the method comprising contacting a sample comprising the PARK7 with

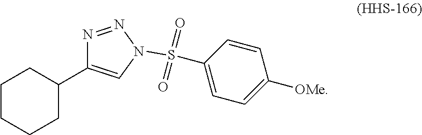 [SMILES: COc1ccc(S(=O)(=O)n2cc(C3CCCCC3)nn2)cc1]

In some embodiments, modulating the formation of RNPGs comprises inhibiting the formation of PGs or increasing the formation of SGs by modulating the multimerization of EDC3, optionally wherein the method comprising contacting a sample comprising the EDC3 with

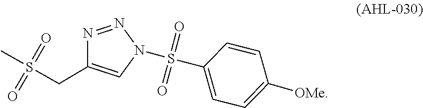 [SMILES: COc1ccc(S(=O)(=O)n2cc(CS(C)(=O)=O)nn2)cc1]

In some embodiments, the presently disclosed subject matter relates to methods for treating a symptom of a disease or infection in a subject in need thereof. In some embodiments, the methods comprise, consist essentially of, or consist of administering to the subject an effective amount of a sulfonyl triazole compound of Formula (I):

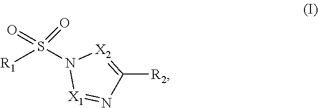

wherein X₁ and X₂ are each CH or N, subject to the proviso that when X₁ is CH, X₂ is N and when X₁ is N, X₂ is CH; R₁ is selected from alkyl, cycloalkyl, aralkyl, aryl, and substituted aryl; and R₂ is selected from alkyl, substituted alkyl, aralkyl, substituted aralkyl, aryl, and substituted aryl, wherein said administrating modulates the formation of pathological ribonucleoprotein granules (RNPGs). In some embodiments, the disease or infection is selected from the group consisting of a viral infection, a cancer, and a neurological disease, optionally frontotemporal dementia or Parkinson's disease.

In some embodiments, the presently disclosed subject matter relates to sulfonyl triazole compounds. In some embodiments, the sulfonyl triazole compound is selected from the group consisting of:

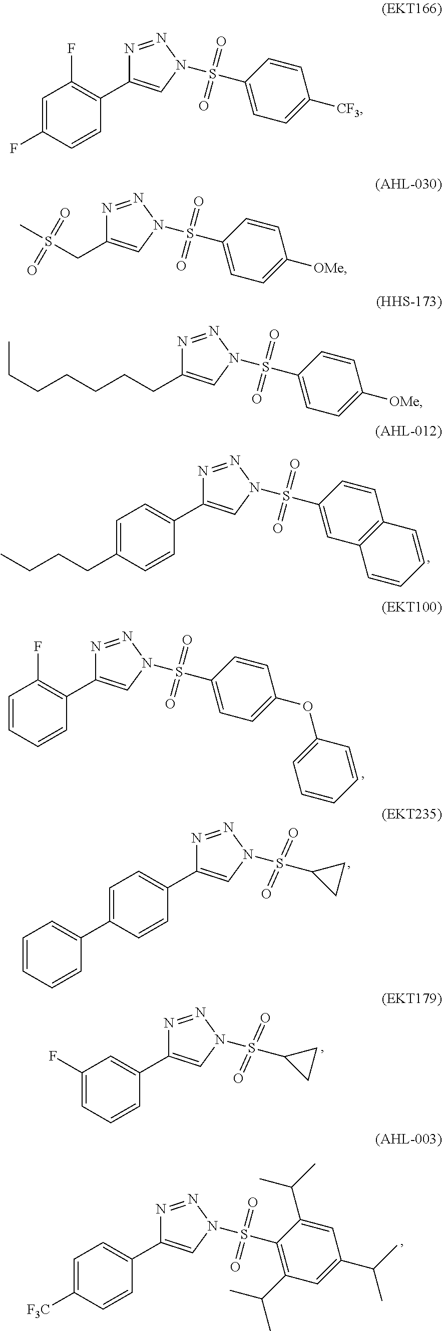

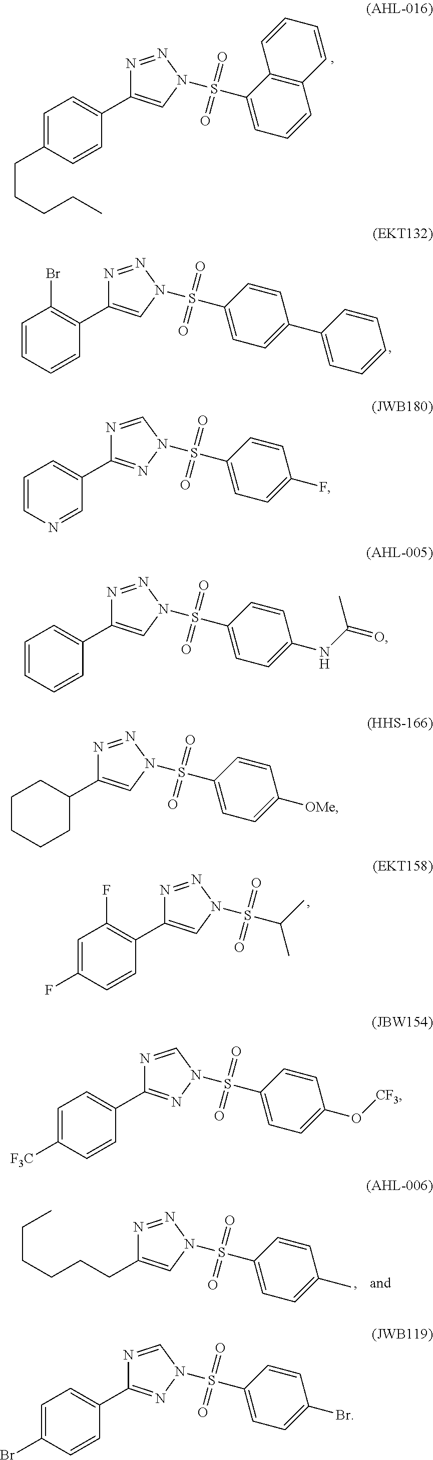

In some embodiments, the sulfonyl triazole compound of claim 15, wherein said compound is selected from the group consisting of AHL-003, EKT166, EKT179, and AHL-030.

The presently disclosed subject matter also relates in some embodiments to pharmaceutical compositions comprising, consisting essentially of, or consisting of a sulfonyl triazole compound as disclosed herein and a pharmaceutical carrier.

The presently disclosed subject matter also relates in some embodiments to compositions for use in treating a symptom associated with a viral infection, cancer, and/or a neurological disease. In some embodiments, the compositions comprise a compound of Formula (I):

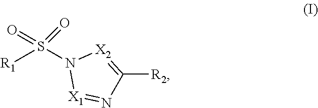

wherein X₁ and X₂ are each CH or N, subject to the proviso that when X₁ is CH, X₂ is N and when X₁ is N, X₂ is CH; R₁ is selected from alkyl, cycloalkyl, aralkyl, aryl, and substituted aryl; and R₂ is selected from alkyl, substituted alkyl, aralkyl, substituted aralkyl, aryl, and substituted aryl, wherein said administrating modulates the formation of pathological ribonucleoprotein granules (RNPGs).

Accordingly, it is an object of the presently disclosed subject matter to provide compositions and methods for compositions for modulating the formation of ribonucleoprotein granules (RNPGs). This and other objects are achieved in whole or in part by the presently disclosed subject matter. Further, objects of the presently disclosed subject matter having been stated above, other objects and advantages of the presently disclosed subject matter will become apparent to those skilled in the art after a study of the following Description, Figures, and EXAMPLES. Additionally, various aspects and embodiments of the presently disclosed subject matter are described in further detail below.

### BRIEF DESCRIPTION OF THE FIGURES

The presently disclosed subject matter will now be described more fully hereinafter with reference to the accompanying Figures, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art. Certain components in the Figures are not necessarily to scale, emphasis instead being placed upon illustrating the principles of the presently disclosed subject matter (in some cases schematically).

FIGS. 1A and 1B. Phenotypic screening for condensate-modulating SuTEx electrophiles. (FIG. 1A) Sulfonyl-triazoles are Tyr/Lys-reactive electrophiles with activity enriched for covalent binding to RNA-binding (RBD) and protein-protein interaction (PPI) domains via sulfur-triazole exchange (SuTEx) chemistry. (FIG. 1B) Phenotypic screening by immunofluorescence (IF) to identify SuTEx electrophile compounds that can function as PB (open circles) and/or SG (solid circles) inhibitors or enhancers. SuTEx ligand-protein interactions of condensate-modulating compounds are deconvoluted by quantitative chemical proteomics.

FIGS. 2A and 2B. SuTEx electrophiles function as different classes of PB and SG modulators. (FIG. 2A) Representative immunofluorescence images depicting effects of SuTEx compound treatments on stress granules (α-G3BP1, red) and P-bodies (α-EDC4, green) in arsenite-treated HeLa and glucose-deprived HEK293T cells, respectively. No treatment [(−)SuTEx] or treatment with a negative control SuTEx compound (JWB180) results in negligible effects on RNP granules in stressed cells. Data are representative of n=2-3 biologically independent replicates. (FIG. 2B) Heat map for P-bodies and stress granules as a percentage of control for the complete SuTEx compound library screened. Heatmap is sorted by PBs. Data are representative of n=2-3 biologically independent replicates. Chemical structures of lead SuTEx hit molecules are shown.

FIGS. 3A-3F. Expanding the ligandable proteome for RNP granule modulation. (FIG. 3A) Heat map depicting Tyr- and Lys-probe-modified sites detected by LC-MS/MS sorted by compound (columns, named below) and hierarchically clustered based on competition ratios (high SR=greater competition) for each probe-modified site (rows). Heat maps are separated by SG-modulators in arsenite-treated HeLa cells (left) and PB-modulators in glucose-deprived HEK293T cells (right). Data are representative of n=2-5 biologically independent replicates. (FIG. 3B) Domain enrichment analysis (Hahm et al., 2020) of liganded sites (SR>2) for SG- (left) and PB-modulating (right) SuTEx compounds. (FIG. 3C) Venn diagram comparing liganded proteins (SR>2) in stressed cells with the reported RNP granule proteome. (FIG. 3D) Target development level of liganded proteins based on the Pharos database (Sheils et al., 2021) Tchem (75); Tbio (212); Tdark (15); and Tclin (9). (FIG. 3E) Plot of fold-enrichment (‘FE’, x-axis, log 2-scale) as a function of P-value (y-axis, -log 10-scale) for Gene Ontology (GO) enrichment analysis (PANTHER; Mi et al., 2013) for biological processes overrepresented in liganded proteins from stressed cells. Significantly changed GO terms are highlighted (red box, log 2 (FE)>2 and −log₁₀(P-value)>10). (FIG. 3F) Top 10 GO terms by FE for SuTEx liganded proteins compared with GO analysis of the RNP granule proteome. Several GO terms were found to be enriched for both SuTEx liganded- and reported RNP granule-proteins.

FIGS. 4A-4F. Discovery of a SuTEx ligand for the hyper-reactive EDC3 Y475 site. (FIG. 4A) Overlap of liganded sites from SuTEx PB modulators with reported RNA granule induction-sensitive lysine and tyrosine sites (RISKYs; Ciancone et al., 2022). The five overlapping sites were further compared to previously reported hyper-RISKY sites (Ciancone et al., 2022) resulting in the identification of the tyrosine-475 (Y475) site on EDC3. (FIG. 4B) Representative immunofluorescence images depicting decreased PBs and increased SGs in AHL-030-treated cells under stress conditions. EKT235 is shown as a negative control. See FIGS. 8 and 9 for additional details. (FIG. 4C) Representative MS1 extracted ion chromatograms (EICs) showing EDC3 Y475 (i.e., amino acid 2 of the peptide sequence IYLCDIGIPQQVFQEVGINYHSPFGCK; SEQ ID NO: 1, which corresponds to amino acids 474-500 of the human enhancer of mRNA-decapping protein 3 isoform 1 polypeptide of GENBANK® Accession No. NP_001135915.1) is liganded by AHL-030 but not EKT235 [SILAC ratio or SR>2 for DMSO vehicle (light, red)/SuTEx fragment ligand (heavy, blue) treatment conditions]. Enrichment (HHS-465/DMSO) and 1:1 (HHS-465/HHS-465) controls are shown. SRs are calculated as the integrated area under the curve ratio of light-to-heavy peptide. Data are representative of n=2-3 biologically independent replicates. (FIG. 4D) Dose dependent inhibition of recombinant EDC3 probe labeling by AHL-030 in situ. EDC3-expressing HEK293T cells were pretreated with varying concentrations of AHL-030 (0.1-100 μM, 2 hours) followed by lysis and probe labeling of soluble proteomes with HHS-465 (100 μM, 1 h, RT). The in situ IC₅₀ for EDC3 probe labeling inhibition by AHL-030 was estimated to be ~6 μM. Comparable expression of recombinant EDC3 across treatment conditions was confirmed by western blots (α-FLAG). Data shown are mean±SEM and representative of n=3 biologically independent replicates. (FIG. 4E) Chemical structures of AHL-030 (left) and inactive control EKT235 (right). (FIG. 4F) Competitive gel-based ABPP analysis verifying AHL-030 (25 μM, 2 h) competition of HHS-465 probe labeling (100 μM, 1 h, RT) of recombinant EDC3-HEK293T expressing cells under glucose-deprived conditions. SAR was demonstrated by lack of activity of other SuTEx compounds tested under the same treatment conditions. Western blots comparing recombinant expression of EDC3 (α-FLAG) and loading controls (α-GAPDH) are shown. Data are representative of n=2 biologically independent replicates.

FIGS. 5A-5C. Discovery of a G3BP1-targeting SuTEx ligand. (FIG. 5A) The SG-modulating SuTEx compounds HHS-166 and EKT179 ligand G3BP1 (Y40; i.e., amino acid 4 of the peptide sequence NSSYVHGGLDSNGKPADAVYGQK; SEQ ID NO: 2, which corresponds to amino acids 37-59 of the human ras GTPase-activating protein-binding protein 1 of GENBANK® Accession No. NP_005745.1) and HSPB1 (K123; i.e., amino acid 9 of the peptide sequence DGVVEITGKHEERQDEHGYISR; SEQ ID NO: 3, which corresponds to amino acids 115-136 of the human heat shock protein family B (small) member 1 polypeptide of GENBANK® Accession No. KAI2546332.1), respectively, as determined by a SILAC ratio (SR)>2 for DMSO vehicle (light, red) compared with SuTEx fragment ligand (heavy, blue) treatment conditions. The lack of binding activity of structurally related compounds (EKT231 and JWB514) provide evidence for structure-activity relationship. A 1:1 SILAC mixing control (HHS-465/HHS-465) is shown and used for normalization. Representative MS1 extracted ion chromatograms (EICs) are shown. (FIG. 5B) The chemical structure of G3BP1 Y40 SuTEx ligand (HHS-166) and matching inactive control compound (EKT231). (FIG. 5C) G3BP1 domains showing the liganded Y40 site located in the NTF2 dimerization domain. Data are representative of n=2-3 biologically independent replicates.

FIGS. 6A and 6B. G3BP1 Y40 mediates the SG-modulating activity of HHS-166. (FIG. 6A) G3BP1 and G3BP2 knockout (G3BP KO) cells rescued with G3BP1 wild-type (WT) or mutants exhibit differential sensitivity to SuTEx fragment treatment in arsenite-induced SG formation (α-G3BP1) as determined by immunofluorescence analysis. Expression of WT recombinant G3BP1 and Y40E mutant but not the Y40F mutant rescued the deficient arsenite-induced SG formation phenotype of G3BP KO cells. Arsenite-mediated SG formation was blocked by pretreatment with HHS-166 but not the inactive control compound EKT231 in G3BP1 WT- but not Y40E-, which lacks a tyrosine for covalent binding at this site, rescued G3BP KO cells. (FIG. 6B) Box and whisker plot of the number of stress granules per cell from immunofluorescence images of G3BP KO rescue with G3BP1 WT/mutant+SuTEx fragment ligand treatments in arsenite-stressed cells from FIG. 6A (“x” indicates the mean; the center line is the median). Pretreatment with HHS-166 in G3BP1 WT-expressing G3BP KO cells resulted in a statistically significant decreases in SG formation (***p<0.001, n.s.: not significant). A two-sample Student's t-test was performed for statistical comparison. Data shown are representative of n=3 biologically independent replicates.

FIG. 7. Chemical structures, designated names, and chemical properties for exemplary SuTEx fragment small molecules screened against P-body and stress granule formation.

FIGS. 8A-8D. (FIG. 8A) Glucose deprivation (15 minutes) induced PB formation in HEK293T cells. (FIG. 8B) Complete set of representative images for the SuTEx PB screen in glucose-deprived HEK293T cells. PBs (green, α-EDC4) and cell nuclei (blue, DAPI) were detected by immunofluorescence (IF). Data are representative of n=2 biologically independent replicates. (FIG. 8C) Quantification of the IF microscopy data from FIG. 8B. Compound treatments (x-axis, 25 μM, 2 hours) are plotted against the number of PBs per glucose-deprived cell (y-axis as % DMSO) and sorted based on PB-modulating activity of SuTEx fragment ligands evaluated. An analysis of variance with Dunnett's multiple comparisons test was performed, comparing against the ‘DMSO’ condition (**** p<0.0001). A separate Welch's t-test confirmed that emetine (50 nM, 2 hours) inhibited PB formation when compared to relevant vehicle treatment (Water; ***p<0.001). Data are representative of n=2-5 biologically independent replicates and are shown as the mean+/−S.E.M. (FIG. 8D) Chemical structures of representative PH-modulating SuTEx fragment ligands.

FIGS. 9A-9D. (FIG. 9A) Arsenite treatment (75 μM, 30 minutes) of HeLa cells induced SG formation. (FIG. 9B) Complete set of representative images for the SuTEx SG screen in arsenite-treated HeLa cells. SGs (red, μ-G3BP1) and cell nuclei (blue, DAPI) were detected by immunofluorescence (IF). Data are representative of n=3 biologically independent replicates. (FIG. 9C) Quantification of the IF microscopy data as depicted in FIG. 9B. Compound treatments (x-axis, 25 μM, 2 hours) are plotted against the number of SGs per cell (y-axis) and sorted by SG-modulating activity of SuTEx fragment ligands tested. An analysis of variance with Dunnett's multiple comparisons test was performed, comparing against the ‘Arsenite’ condition (*p<0.05, **p<0.01, ***p<0.001, ****p<0.0001). Data are representative of n=3 biologically independent replicates and are shown as the mean+/−S.E.M. (FIG. 9D) Chemical structures of representative SG-modulating SuTEx fragment compounds.

FIG. 10. Competitive ABPP SILAC workflow to detect liganded sites for SuTEx modulators of RNP granules in stressed cells. Chemical structure of the global Tyr/Lys-reactive probe HHS-465 used for chemical proteomics is shown.

FIG. 11. Distribution of the Tyr/Lys (Y/K) ratio of liganded sites for each respective SuTEx fragment hit.

FIG. 12. Plot of HHS-465 probe modified sites as a function of the SILAC ratio (mean SR of Tyr and Lys sites detected in at least 2 biological replicates) from DMSO vehicle (light)/AHL-030 (heavy) treatments in glucose-deprived HEK293T cells. The liganded sites are highlighted by red circles and solid arrows. The liganded site (Y475) on the PB protein EDC3 is denoted by a green circle and a dotted arrow. Quantified sites above the SR threshold of 2 (denoted by dotted line) that were not designated as liganded (black circles) displayed high SR variability between replicates.

FIG. 13. Representative MS1 extracted ion chromatograms (EICs) showing HNRPK Y72 was liganded by EKT166 but not HHS-166 [SILAC ratio or SR>2 for DMSO vehicle (light, red)/SuTEx fragment ligand (heavy, blue) treatment conditions]. A 1:1 (HHS-465/HHS-465) mixing control is shown.

FIG. 14. Plot of HHS-465 probe modified sites as a function of the SILAC ratio (mean SR of Tyr and Lys sites detected in at least 2 biological replicates) from DMSO vehicle (light) I HHS-166 (heavy) treatments in arsenite-treated HeLa cells. The liganded sites are highlighted by red circles and solid arrows. The liganded site (Y40) on the SG protein G3BP1 is denoted by a green circle and a dotted arrow. Quantified sites above the SR threshold of 2 (denoted by dotted line) that were not designated as liganded (black circles) displayed high SR variability between replicates.

FIGS. 15A-15C. Dose-dependent inhibition of arsenite-induced SGs by HHS-166. (FIG. 15A) HeLa cells were treated with HHS-166 at the indicated concentrations (1-25 μM, 2 hours) followed by sodium arsenite stress (75 μM, 30 minutes) and dose-dependent effects on SG formation (α-G3BP 1) were quantified by immunofluorescence analysis. (FIG. 15B) Box and whisker plot of the number of SGs per cell from immunofluorescence images of G3BP1+SuTEx fragment ligand treatments in arsenite-stressed cells from FIG. 15A (“x” indicates the mean; the center line is the median). Treatments with 12.5 μM or higher concentrations of HHS-166 resulted in a statistically significant decreases in SG formation (***p<0.001, n.s.: not significant). A two-sample Student's t-test was performed for statistical comparison. (FIG. 15C) The average number of SGs per cell from FIG. 15B was plotted as a function of HHS-166 concentrations from cellular treatments to estimate the EC₅₀ (~8 μM) for arsenite-induced SG inhibition in situ. Data shown are representative of n=2 biologically independent replicates.

### DETAILED DESCRIPTION

Stress granules (SGs) and processing-bodies (PBs, P-bodies) are ubiquitous and widely studied ribonucleoprotein (RNP) granules involved in cellular stress response, viral infection, and the tumor microenvironment. While proteomic and transcriptomic investigation of SGs and PBs have provided insights into molecular composition, chemical tools to probe and modulate RNP granules remain lacking.

PBs and SGs share many common features, but their functional roles, while not fully elucidated, diverge substantially in the pathogenesis of a variety of diseases, such as cancer and viral infection. SGs have been implicated in frontotemporal dementia, cancer, and viral infection (see e.g., Anderson et al., 2015; Marmor-Kollet et al., 2020; Reineke & Lloyd, 2015; Mackenzie et al., 2017; Baradaran-Heravi et al., 2020; Hans et al., 2020). For example, viruses, such as SARS-CoV-2, MERS, TGEV, and MHV, are known to interact with PKR and PERK, eIF2a kinases, and other critical SG proteins in order to modulate the formation of SGs and to hijack cellular translational machinery (Garcia et al., 2006; Reineke & Lloyd, 2015; Gordon et al., 2020). Cancers can become resistant to radiation or chemotherapeutics caused in part by the formation of pro-survival SGs (Kwon et al., 2007; Arimoto et al., 2008; Fournier et al., 2010; Fujimura et al., 2012; Kaehler et al., 2014). Much less is known about the disease relevance of PBs, but these granules are known to sequester translationally repressed and AU-rich RNA, which could provide a unique mechanism for pharmacological regulation of such transcripts (Franks & Lykke-Andersen, 2007; Takahashi et al., 2011; Garcia-Maurino et al., 2017; Hubstenberger et al., 2017). PBs may be critical for the immune response, as it has been shown that PB nucleation by DCP1a and EDC4 is critical for the production of IL-6 in macrophages (Seto et al., 2015).

Recently, PBs have been shown to be regulated by Alpha-synuclein, an aggregating protein genetically linked with Parkinson's disease (Hallacli et al., 2022). Overall, however, the function of PBs in terms of disease remains an on-going area of investigation. Regardless, the ability to pharmacologically regulate these RNAGs has recently gained significant interest.

Currently, there is a lack of non-toxic and easily tunable pharmacological modulators for RNAG formation. Emetine and cycloheximide can inhibit and even dissociate PBs, but these drugs function by shutting off the translational machinery of a cell, leading to significant toxicity (Cougot et al., 2004; Kedersha & Anderson, 2007; Colombrita et al., 2009). Pateamine A, silvestrol, rocaglamide A, and hippuristanol inhibit eIF4A, interfering with the eIF4F complex, leading to defects in the pre-initiation complex that is critical for translation, resulting in the formation of RNAGs (Bordeleau et al., 2005; Low et al., 2005; Kedersha & Anderson, 2007). Vinca alkaloids, such as vinblastine, also form PBs by binding tubulin and inhibiting the assembly of microtubules, which prevents cells from undergoing mitosis (Aizer et al., 2008; Ayache et al., 2015). Additionally, many inhibitor studies have been performed in bacterial systems, where the machinery used to form the RNAGs is significantly different from that of mammalian systems (Luo et al., 2018).

A variety of cancer studies have shown that chemotherapeutics, such as bortezomib and 5-fluorouracil, can lead to the formation of pro-survival SGs in solid tumors, which can contribute to chemoresistance (Fournier et al., 2010; Kaehler et al., 2014; Anderson et al., 2015). A high-throughput screen of nearly 10,000 compounds found that planar, aromatic compounds could prevent TDP-43 aggregation in SGs, which is believed to be critical for the pathogenesis of neurodegenerative diseases, such as amyotrophic lateral sclerosis. However, the mechanism of action for these compounds that directly contributes to condensate assembly is unclear and could prove difficult to elucidate (Sreedharan et al., 2008; Fang et al., 2019). Identification of a novel, easily tunable class of compounds that can be mechanistically unraveled and can modulate RNAGs could prove useful for studying RNAG biology and disease relevance.

Summarily, undesirable changes in SG and PB biological activities are associated with various pathological consequences in many neurological, immunological, and infectious diseases (Bloch et al., 2013; Lloyd, 2013; Vanderweyde et al., 2013; Buchan, 2014), as well as the initiation and progression of tumors and cancer (Anderson et al., 2015).

As disclosed herein, an immunofluorescence-based phenotypic screen was combined with chemoproteomics to identify sulfonyl-triazoles (SuTEx) capable of preventing or inducing SG and PB formation through liganding of tyrosine and lysine sites in stressed cells. Liganded sites were enriched for RNA-binding and protein-protein interaction domains, including several sites found in RNP granule-forming proteins. Among these, we functionally validate G3BP1 Y40, located in the NTF2 dimerization domain, as a ligandable site that can disrupt arsenite-induced SG formation in cells. In summary, we present a chemical strategy for the systematic discovery of condensate-modulating covalent small molecules.

### I. Definitions

Unless otherwise defined, all technical and scientific terms used herein have the same meaning as commonly understood by one of ordinary skill in the art to which the presently described subject matter belongs.

Throughout the specification and claims, a given chemical formula or name shall encompass all active optical and stereoisomers, as well as racemic mixtures where such isomers and mixtures exist.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a protein” refers to one or more proteins, including a plurality of the same protein. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As used herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

In some embodiments, the terms “fragment”, “segment”, or “subsequence” as used herein refers to a portion of an amino acid sequence, comprising at least one amino acid, or a portion of a nucleic acid sequence comprising at least one nucleotide. Thus, in some embodiments, the terms “fragment”, “segment”, and “subsequence” are used interchangeably herein. In some embodiments, the term “fragment” refers to a compound (e.g., a small molecule compound, such as a small molecule comprising a purine scaffold) that can react with a reactive amino acid residue (e.g., a reactive cysteine) to form an adduct comprising a modified amino acid residue. Thus, in some embodiments, the terms “fragment” and “ligand” are used interchangeably. In some embodiments, the term “fragment” refers to that portion of a ligand that remains covalently attached to the reactive amino acid residue.

As used herein, a “ligand” is a compound (e.g., a purine-based compound) that specifically binds to a target compound or molecule, such as a reactive nucleophilic amino acid residue in a protein. In some embodiments, the ligand can bind to the target covalently. A ligand “specifically binds to” or “is specifically reactive with” a compound (e.g., a reactive amino acid residue) when the ligand functions in a binding reaction which is determinative of the presence of the compound in a sample of heterogeneous compounds.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules can be ionized and detected by any suitable means known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” can refer to the application of mass spectrometry to protein analysis. In some embodiments, electrospray ionization (ESI) and matrix-assisted laser desorption/ionization (MALDI) can be used in this context. In some embodiments, intact protein molecules can be ionized by the above techniques, and then introduced to a mass analyzer. Alternatively, protein molecules can be broken down into smaller peptides, for example, by enzymatic digestion by a protease, such as trypsin. Subsequently, the peptides are introduced into the mass spectrometer and identified by peptide mass fingerprinting or tandem mass spectrometry.

As used herein, the term “mass spectrometer” is used to refer an apparatus for performing mass spectrometry that includes a component for ionizing molecules and detecting charged molecules. Various types of mass spectrometers can be employed in the methods of the presently disclosed subject matter. For example, whole protein mass spectroscopy analysis can be conducted using time-of-flight (TOF) or Fourier transform ion cyclotron resonance (FT-ICR) instruments. For peptide mass analysis, MALDI time-of-flight instruments can be employed, as they permit the acquisition of peptide mass fingerprints (PMFs) at high pace. Multiple stage quadrupole-time-of-flight and the quadrupole ion trap instruments can also be used.

The terms “high throughput protein identification,” “proteomics” and other related terms are used herein to refer to the processes of identification of a large number or (in some cases, all) proteins in a certain protein complement. Post-translational protein modifications and quantitative information can also be assessed by such methods. One example of “high throughput protein identification” is a gel-based process that includes the pre-fractionation and purification of proteins by one-dimensional protein gel electrophoresis. The gel can then be fractionated into several molecular weight fractions to reduce sample complexity, and proteins can be in-gel digested with trypsin. The tryptic peptides are extracted from the gel, further fractionated by liquid chromatography, and analyzed by mass spectrometry. In another approach, a sample can be fractionated without using the gels, for example, by protein extraction followed by liquid chromatography. The proteins can then be digested in-solution, and the proteolytic fragments further fractionated by liquid chromatography and analyzed by mass spectrometry.

As used herein, the term “Western blot,” which can be also referred to as “immunoblot”, and related terms refer to an analytical technique used to detect specific proteins in a sample. The technique uses gel electrophoresis to separate the proteins, which are then transferred from the gel to a membrane (typically nitrocellulose or PVDF) and stained, in membrane, with antibodies specific to the target protein.

The expression “stable isotope labeling by amino acids in cell culture” (SILAC) is used herein to refer to an approach for incorporation of a label into proteins for mass spectrometry (MS)-based quantitative proteomics. SILAC comprises metabolic incorporation of a given “light” or “heavy” form of the amino acid into the proteins. For example, SILAC comprises the incorporation of amino acids with substituted stable isotopic nuclei (e.g. deuterium, ¹³C, ¹⁵N). In an illustrative SILAC experiment, two cell populations are grown in culture media that are identical, except that one of them contains a “light” and the other a “heavy” form of a particular amino acid (for example, ¹²C and ¹³C labeled L-lysine, respectively). When the labeled analog of an amino acid is supplied to cells in culture instead of the natural amino acid, it is incorporated into all newly synthesized proteins. After a number of cell divisions, each instance of the amino acid is replaced by its isotope-labeled analog. Since there is little chemical difference between the labeled amino acid and the natural amino acid isotopes, the cells behave substantially similar to the control cell population grown in the presence of a normal amino acid.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans, and nonhuman primates such as chimpanzees and other apes and monkey species; farm animals such as cattle, sheep, pigs, goats, and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains proteins, cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Exemplary Compositions and Methods of Use Thereof

In some embodiments, the presently disclosed subject matter relates to sulfonyl triazole compounds, compositions comprising, consisting essentially of, or consisting of the same, pharmaceutical compositions thereof, and methods for using the disclosed compounds and compositions for modulating the formation of ribonucleoprotein granules (RNPGs), treating a symptom of a disease or infection in a subject in need thereof, which in some embodiments can be a symptom associated with a viral infection, a tumor and/or a cancer, and/or a neurological disease.

### II.A. Compositions

In some embodiments, a sulfonyl triazole compound of the presently disclosed subject matter has a structure of Formula (I):

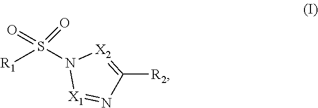

wherein X₁ and X₂ are each CH or N, subject to the proviso that when X₁ is CH, X₂ is N and when X₁ is N, X₂ is CH; R₁ is selected from alkyl, cycloalkyl, aralkyl, aryl, and substituted aryl; and R₂ is selected from alkyl, substituted alkyl, aralkyl, substituted aralkyl, aryl, and substituted aryl. In some embodiments, X₁ is N and X₂ is CH. In some embodiments, R₁ is selected from the group consisting of isopropyl, cyclopropyl, naphthyl, pyridyl, phenyl, and substituted phenyl. In some embodiments, R₁ is substituted phenyl, wherein said substituted phenyl is phenyl substituted with one or more substituents selected from the group consisting of cyano, halo, alkyl, alkoxy, perfluoroalkyl, perfluoroalkoxy, phenyl, and —NH(═O)-alkyl, optionally wherein substituted phenyl is phenyl substituted with one or more substituents selected from the group consisting of cyano, bromo, fluoro, methyl, isopropyl, methoxy, trifluoromethyl, trifluoromethoxy, phenyl, and —NH(═O)-methyl. In some embodiments, R₂ is selected from the group consisting of alkyl, sulfonyl-substituted alkyl, cycloalkyl, phenyl, pyridyl, and substituted phenyl. In some embodiments, R₂ is substituted phenyl, wherein said substituted phenyl is phenyl substituted with one or more substituents selected from the group consisting of halo, alkyl, perfluoroalkyl, alkoxy, and phenyl, optionally, fluoro, bromo, chloro, butyl, pentyl, trifluoromethyl, methoxy, and phenyl. In some embodiments, R₂ is selected from the group consisting of heptyl, hexyl, cyclohexyl, —CH₂—S(═O)₂—CH₃, pyridyl, and phenyl.

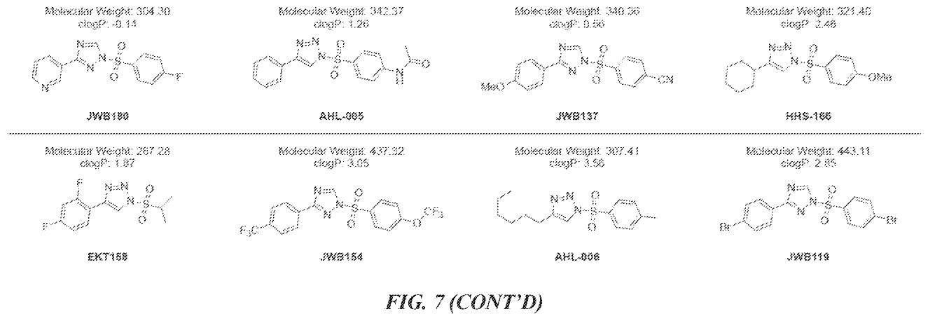

Representative examples of sulfonyl triazole compound of the presently disclosed subject matter of Formula (I) include EKT231, EKT166, AHL-030, HHS-173, AMC-001, AHL-012, EKT100, EKT235, EKT179, AHL-003, AHL-016, EKT132, JWB180, AHL-005, JWB137, HHS-166, EKT158, JWB154, AHL-006, and JWB119, the structures of which are shown in FIG. 7.

### II.B. Pharmaceutical Compositions and Administration

The presently disclosed subject matter also relates in some embodiments to pharmaceutical compositions comprising, consisting essentially of, or consisting of one or more sulfonyl triazole compounds of the presently disclosed subject matter and a pharmaceutically acceptable carrier, diluent, and/or excipient.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means. As such, in some embodiments the presently disclosed compositions are administered by injecting the composition subcutaneously, intraperitoneally, into adipose tissue, and/or intramuscularly into the subject.

In accordance with some embodiments, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isostearyl alcohols, polyoxyethylene sorbitol and sorbitan esters, microcrystalline cellulose, aluminum metahydroxide, bentonite, kaolin, agar-agar and tragacanth, or mixtures of these substances, and the like.

In some embodiments wherein a composition of the presently disclosed subject matter is desired to induce an immune response, the compositions of the presently disclosed subject matter can further comprise an adjuvant. In some embodiments, the at least one adjuvant is selected from the group consisting of montanide ISA-51 (Seppic, Inc.), QS-21 (Aquila Pharmaceuticals, Inc.), tetanus helper peptides, GM-CSF, cyclophosamide, bacillus Calmette-Guerin (BCG), Corynebacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, peptides, oil emulsions, dinitrophenol, diphtheria toxin (DT).

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracistemally, intravaginally, intraperitoneally, locally (e.g., with powders, ointments, or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion, and catheter instillation into the vasculature), peri- and intra-target tissue injection, subcutaneous injection or deposition including subcutaneous infusion, intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1985; Gennaro, 1990; or Gennaro, 2003; each of which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient.

The presently disclosed subject matter also related to methods for using the compositions of the presently disclosed subject matter for various purposes. For example, in some embodiments the presently disclosed subject matter also relates to methods for treating and/or preventing diseases, disorders, and/or conditions associated with inflammation.

### II.C. Dosages

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated, such as but not limited to a reduction in scarring and/or fibrosis, particularly as compared to the same subject had the subject not received the composition). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### II.D. Routes of Administration

Suitable methods for administration of the compositions of the presently disclosed subject matter include, but are not limited to intravenous administration, oral delivery, and delivery directly to a target tissue or organ (e.g., a topical application and/or a site of injury such as but not limited to a muscle injury). Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated. By way of example and not limitation, in some embodiments a composition of the presently disclosed subject matter is administered to the subject via a route selected from the group consisting of intraperitoneal, intramuscular, intravenous, and intranasal, or any combination thereof.

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethylene glycol, hyaluronic acid, ethanol, cyclodextrins, modified cyclodextrins (i.e., sulfobutyl ether cyclodextrins), etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like.

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of a liquid, suspension, cream, ointment, lotion, or the like, preferably in unit dosage form suitable for single administration of a precise dosage. The compositions can in some embodiments include one or more pharmaceutically acceptable carriers and, in addition, may include other pharmaceutical agents, adjuvants, diluents, buffers, etc.

### II.E. Methods of Treatment and Other Uses

In some embodiments, the presently disclosed subject matter relates to methods for modulating the formation of ribonucleoprotein granules (RNPGs). In some embodiments, the methods comprise, consist essentially of, or consist of contacting a sample comprising a protein with a sulfonyl triazole compound, thereby covalently modifying the protein. In some embodiments, the RNPG is a stress granule (SG) or a processing body (PB) and/or wherein the sulfonyl triazole compound is capable of covalently modifying an RNA granule induction-sensitive lysine and tyrosine site (RISKY) in the protein.

In some embodiments of the presently disclosed methods, the sulfonyl triazole compound has a structure of Formula (I):

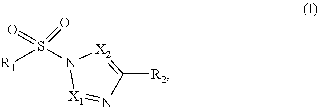

wherein X₁ and X₂ are each CH or N, subject to the proviso that when X₁ is CH, X₂ is N and when X₁ is N, X₂ is CH; R₁ is selected from alkyl, cycloalkyl, aralkyl, aryl, and substituted aryl; and R₂ is selected from alkyl, substituted alkyl, aralkyl, substituted aralkyl, aryl, and substituted aryl. In some embodiments, X₁ is N and X₂ is CH. In some embodiments, R₁ is selected from the group consisting of isopropyl, cyclopropyl, naphthyl, pyridyl, phenyl, and substituted phenyl. In some embodiments, R₁ is substituted phenyl, wherein said substituted phenyl is phenyl substituted with one or more substituents selected from the group consisting of cyano, halo, alkyl, alkoxy, perfluoroalkyl, perfluoroalkoxy, phenyl, and —NH(═O)-alkyl, optionally wherein substituted phenyl is phenyl substituted with one or more substituents selected from the group consisting of cyano, bromo, fluoro, methyl, isopropyl, methoxy, trifluoromethyl, trifluoromethoxy, phenyl, and —NH(═O)-methyl. In some embodiments, R₂ is selected from the group consisting of alkyl, sulfonyl-substituted alkyl, cycloalkyl, phenyl, pyridyl, and substituted phenyl. In some embodiments, R₂ is substituted phenyl, wherein said substituted phenyl is phenyl substituted with one or more substituents selected from the group consisting of halo, alkyl, perfluoroalkyl, alkoxy, and phenyl, optionally, fluoro, bromo, chloro, butyl, pentyl, trifluoromethyl, methoxy, and phenyl. In some embodiments, R₂ is selected from the group consisting of heptyl, hexyl, cyclohexyl, —CH₂—S(═O)₂—CH₃, pyridyl, and phenyl.

In some embodiments of the presently disclosed methods, the modulating inhibits the formation of PB and/or SG. Exemplary sulfonyl triazole compounds of the presently disclosed subject matter that can be employed to inhibit the formation of PB and/or SG include, but are not limited to compounds selected from the group consisting of:

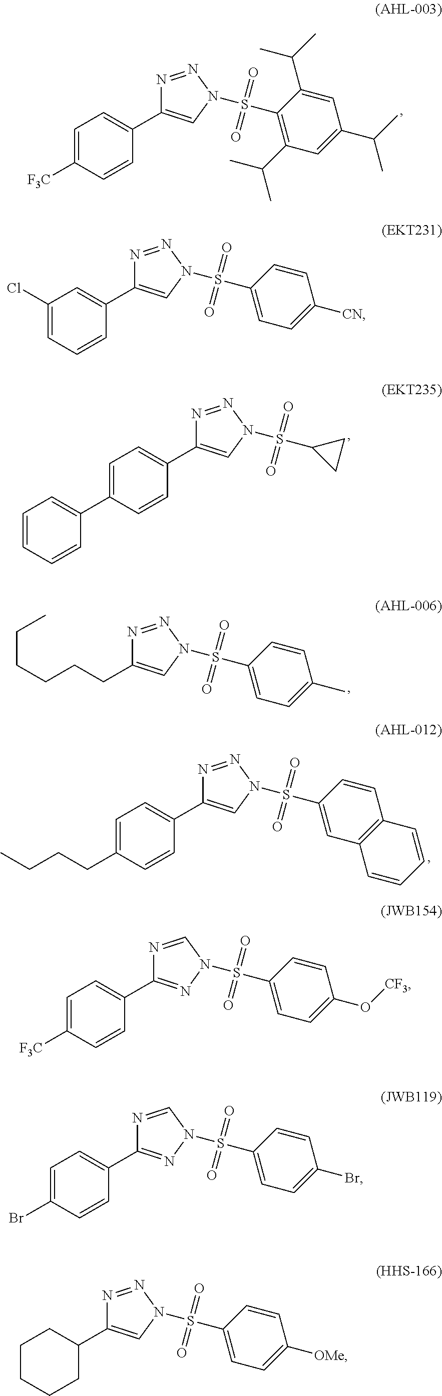

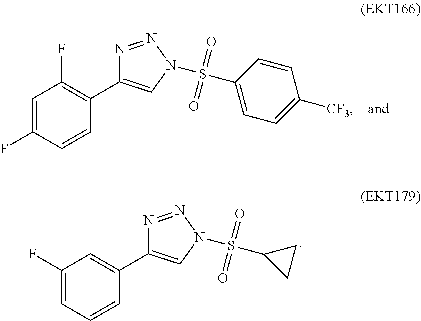 [SMILES: O=S(=O)(c1ccc(C(F)(F)F)cc1)n1cc(-c2ccc(F)cc2F)nn1 ; "and" ; O=S(=O)(C1CC1)n1cc(-c2cccc(F)c2)nn1]

Alternatively, in some embodiments the modulation induces the formation of PB and/or SG. Exemplary sulfonyl triazole compounds of the presently disclosed subject matter that can be employed to induce the formation of PB and/or SG include, but are not limited to the following compounds:

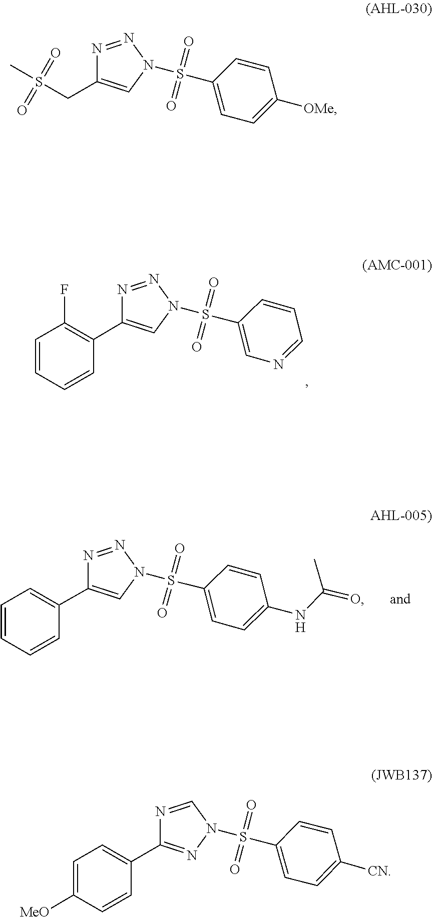 [SMILES: COc1ccc(S(=O)(=O)n2cc(CS(C)(=O)=O)nn2)cc1 ; O=S(=O)(c1cccnc1)n1cc(-c2ccccc2F)nn1 ; CC(=O)Nc1ccc(S(=O)(=O)n2cc(-c3ccccc3)nn2)cc1 ; COc1ccc(-c2ncn(S(=O)(=O)c3ccc(C#N)cc3)n2)cc1]

In some embodiments, the presently disclosed subject matter relates to modulating the formation of RNPGs by inhibiting the multimerization of G3BP stress granule assembly factor 1 (G3BP1). As used herein, the term “G3BP1” refers to a genetic locus or any gene product thereof (e.g., a nucleic acid or an amino acid gene product) that corresponds to the human G3BP stress granule assembly factor 1 (G3BP1), transcript variant 1 of GENBANK® Accession No. NM_005754.4, which encodes the polypeptide set forth in GENBANK® Accession No. NP_005745.1, the human G3BP stress granule assembly factor 1 (G3BP1), transcript variant 2 of GENBANK® Accession No. NM_198395.2, which encodes the polypeptide set forth in GENBANK® Accession No. NP_938405.1, biologically active fragments thereof, homologs thereof, and orthologs thereof from other species. In some embodiments, the presently disclosed methods comprise, consist essentially of, or consist of contacting a sample comprising G3BP1 with a composition comprising, consisting essentially of, or consisting of sulfonyl triazole compound HHS-166:

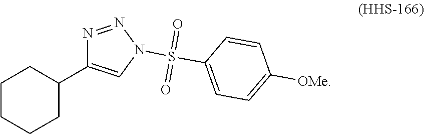 [SMILES: COc1ccc(S(=O)(=O)n2cc(C3CCCCC3)nn2)cc1]

In some embodiments, the presently disclosed subject matter relates to modulating the formation of RNPGs comprises inhibiting the multimerization of Parkinsonism associated deglycase (PARK7) polypeptides. As used herein, the term “PARK7” refers to a genetic locus or any gene product thereof (e.g., a nucleic acid or an amino acid gene product) that corresponds to the human Parkinsonism associated deglycase of GENBANK® Accession No. NM_007262.5, which encodes the polypeptide set forth in GENBANK® Accession No. NP_009193.2, biologically active fragments thereof, homologs thereof, and orthologs thereof from other species. In some embodiments, inhibiting the multimerization of PARK7 polypeptides can be accomplished by contacting a sample comprising the PARK7 with a composition comprising, consisting essentially of, or consisting of sulfonyl triazole compound HHS-166 (structure shown above).

In some embodiments, the presently disclosed subject matter relates to modulating the formation of RNPGs by inhibiting the formation of PGs or increasing the formation of SGs. In some embodiments, this is accomplished by modulating the multimerization of enhancer of mRNA decapping 3 (EDC3) polypeptides. As used herein, the term “EDC3” refers to a genetic locus or any gene product thereof (e.g., a nucleic acid or an amino acid gene product) that corresponds to the human enhancer of mRNA decapping 3 of GENBANK® Accession No. NM_001142443.3, which encodes the polypeptide set forth in GENBANK® Accession No. NP_001135915.1, biologically active fragments thereof, homologs thereof, and orthologs thereof from other species. In some embodiments, modulating the multimerization of EDC3 polypeptides can be accomplished by contacting a sample comprising the an EDC3 polypeptide with a composition comprising, consisting essentially of, or consisting of a sulfonyl triazole compound of the presently disclosed subject matter. In some embodiments, the method comprises, consists essentially of, or consists of contacting a sample comprising the EDC3 with a composition comprising, consisting essentially of, or consisting of sulfonyl triazole compound AHL-030:

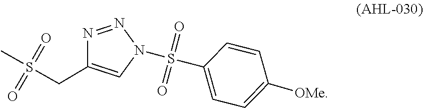 [SMILES: COc1ccc(S(=O)(=O)n2cc(CS(C)(=O)=O)nn2)cc1]

In some embodiments, the presently disclosed subject matter also relates to methods for treating symptoms of diseases and/or infections in subjects in need thereof. In some embodiments, the methods comprise, consist essentially of, or consist of administering to the subject an effective amount of a sulfonyl triazole compound of Formula (I):

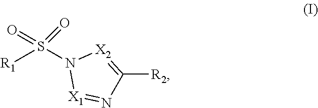

wherein X₁ and X₂ are each CH or N, subject to the proviso that when X₁ is CH, X₂ is N and when X₁ is N, X₂ is CH; R₁ is selected from alkyl, cycloalkyl, aralkyl, aryl, and substituted aryl; and R₂ is selected from alkyl, substituted alkyl, aralkyl, substituted aralkyl, aryl, and substituted aryl, wherein said administrating modulates the formation of pathological ribonucleoprotein granules (RNPGs).

In some embodiments, the disease or infection is selected from a viral infection, a tumor and/or a cancer, and/or a neurological disease. In some embodiments, the neurological disease is frontotemporal dementia or Parkinson's disease.

With respect to the treatment methods disclosed herein, it is also possible to employ a sulfonyl triazole compound of the presently disclosed subject matter or any combination thereof as part of a combination therapy for diseases and/or infections in subjects in need thereof, including but not limited to viral infections, tumors and/or cancers, and/or neurological diseases. By way of example and not limitation, when a microbial or viral infection is to be treated with a composition or method of the presently disclosed subject matter, additional antimicrobial and/or antiviral treatments can also be administered to a subject. Depending on the nature of the infection, one of ordinary skill in the art would be aware of antimicrobial and/or antiviral treatments that can be part of a combination therapy with the sulfonyl triazole compounds of the presently disclosed subject matter.

Similarly, it is also possible to employ a sulfonyl triazole compound of the presently disclosed subject matter or any combination thereof as part of a combination therapy for tumors and/or cancers. As such, in some embodiments the presently disclosed methods employ a combination therapy with one or more sulfonyl triazole compounds in combination with a chemotherapeutic agent. Examples of chemotherapeutic agents that can be used in accordance with the presently disclosed subject matter include, but are not limited to, platinum coordination compounds such as cisplatin, carboplatin or oxalyplatin; taxane compounds, such as paclitaxel or docetaxel; topoisomerase I inhibitors such as camptothecin compounds for example irinotecan or topotecan; topoisomerase II inhibitors such as anti-tumor podophyllotoxin derivatives for example etoposide or teniposide; anti-tumor vinca alkaloids for example vinblastine, vincristine or vinorelbine; anti-tumor nucleoside derivatives for example 5-fluorouracil, gemcitabine or capecitabine; alkylating agents, such as nitrogen mustard or nitrosourea for example cyclophosphamide, chlorambucil, carmustine or lomustine; anti-tumor anthracycline derivatives for example daunorubicin, doxorubicin, idarubicin or mitoxantrone; HER2 antibodies for example trastuzumab; estrogen receptor antagonists or selective estrogen receptor modulators for example tamoxifen, toremifene, droloxifene, faslodex or raloxifene; aromatase inhibitors, such as exemestane, anastrozole, letrazole and vorozole; differentiating agents such as retinoids, vitamin D and retinoic acid metabolism blocking agents (RAMBA) for example accutane; DNA methyl transferase inhibitors for example azacytidine; kinase inhibitors for example flavoperidol, imatinib mesylate or gefitinib; famesyltransferase inhibitors; HDAC inhibitors; other inhibitors of the ubiquitin-proteasome pathway for example VELCADE® (Millennium Pharmaceuticals, Cambridge, Massachusetts, United States of America); or YONDELIS (Johnson & Johnson, New Brunswick, New Jersey, United States of America). Various formulations that are within the skill of the ordinary artisan can be employed in the compositions and methods of the presently disclosed subject matter.

Additionally, combination treatments with sulfonyl triazole compounds of the presently disclosed subject matter can be employed to treat neurological diseases. In some embodiments, the neurological disease is frontotemporal dementia or Parkinson's disease. Therapeutic agents that can be employed in combination with the sulfonyl triazole compounds of the presently disclosed subject matter to treat neurological diseases are also within the knowledge of one of ordinary skill in the art.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the Examples

Cell culture. HEK293T and HeLa normal and SILAC cells were cultured as previously described in Ciancone et al., 2022.

SuTEx compound treatments. Prior to treatments, cells were washed twice with warm phosphate-buffered saline (PBS) and exchanged with serum-free DMEM (supplemented with 1% L-glutamine) for at least 30 minutes at 37° C. with 5% CO₂. Compounds were then added as 1000×DMSO stocks (10 μL for 10 cm dishes and 3 μL for 6-well dishes), plates were mixed gently, and cells were incubated for two hours. All compounds were thoroughly vortexed and sonicated for five minutes prior to addition to ensure complete solvation. For microscopy experiments, cultures were seeded at 150,000 cells in 3 mL of DMEM in 6-well THERMO SCIENTIFIC™ BIOLITE™ brand microwell plates (Catalog No. 12556004; Thermo Fisher Scientific Inc., Waltham, Massachusetts. United States of America). For collection, media was aspirated, and cells were washed once with cold PBS before scraping from plates. Cells were centrifuged at 500×g at 4° C. for 5 minutes and supernatant was removed. Cell pellets were resuspended in cold PBS (1 mL) and centrifuged at 1,400×g at 4° C. for 3 minutes. Supernatant was again removed, and pellets were then snap-frozen in liquid nitrogen and stored at −80° C. until needed.

Preparation of SILAC proteomes for LC-MS/MS analysis. SILAC cell proteomes were fractionated to collect the supernatant and normalized to 2.3 mg/mL in PBS. ‘Heavy’ and ‘light’ proteomes were aliquoted (427 μL) and probe-modified proteins were conjugated to desthiobiotin-PEG-3-azide using copper-catalyze azide-alkyne cycloaddition, trypsinized into peptides using Trypsin/Lys-C Mix (Catalog No. V5071; Promega Corporation, Madison, Wisconsin, United States of America), enriched using PIERCE™ Avidin Agarose brand beads (Catalog No. 20219; Thermo Fisher Scientific Inc., Waltham, Massachusetts. United States of America), and prepared for nano-electrospray ionization-LC-MS/MS analysis as previously described in Hahm et al., 2020.

Immunofluorescence. Immunofluorescence (IF) of HEK293T cells was performed as previously described in Ciancone et al., 2022. HeLa cells were seeded at 9.0×10⁴ in a 24-well plate with 12-mm glass coverslips. The next day, the cells were treated with 25 μM SuTEx probe for 2 hours in DMEM medium without FBS. The cells were then treated with 75 μM NaAsO₂ for 30 minutes and fixed with 3% paraformaldehyde in PBS for 15 minutes at room temperature (RT) and washed with PBS twice. The fixed cells were permeabilized with PBST (0.1% Triton X-100) for 15 minutes at room temperature and blocked with 5% goat serum in PBST for 30 minutes at RT. The cells were incubated with anti-G3BP1 antibody H-10 (Catalog No. sc-365338 at 1:200 dilution; Santa Cruz Biotechnology, Inc., Dallas, Texas, United States of America) for 2 hours at RT and washed with PBST (0.1% Tween-20) for 5 minutes three times. Next, cells were incubated with goat anti-mouse Alexa 594 antibody (Catalog No. 115-585-003 at 1:800 dilution; Jackson ImmunoResearch, West Grove, Pennsylvania, United States of America) for 30 minutes at RT. The cells were washed twice with PBST, stained with Hoechst 33342 (Catalog No. H3570 at 111 g/mL; Thermo Fischer Scientific) in PBST for 5 minutes, and washed with PBS twice for 5 minutes. The coverslips were mounted in PROLONG™ Gold AntiFade Mountant (Catalog No. P36930, Thermo Fisher Scientific) and imaged using NIS Elements AR software and a Nikon Eclipse Ti microscope equipped with a ×100 objective and CMOS camera. Images used for direct comparison were acquired using standardized illumination and exposure settings and displayed with identical lookup table settings. A CellProfiler pipeline was developed to quantify the number of stress granules under each condition. Briefly, cells were recognized as blue⁺ (nuclei) red⁺ (G3BP1) objects, and the red⁺ granules that meet the shape and size threshold were identified and counted. Cells on the border of linages were excluded from the analysis for more accurate quantification.

G3BP KO and rescue studies. U20S G3BP1/2 double knockout (G3BP KO) cell-line was a kind gift from the C. Brangwynne lab (Princeton University, Princeton, New Jersey, United States of America) and generated as previously described in Kedersha et al., 2016. G3BP KO cells were seeded at 9.0×10⁴ in a 24-well plate with 12-mm glass coverslips. Next day, the cells were transfected with pcDNA5-G3BP1(WTN40F/Y40E) vectors with 1 μg/mL tetracycline and LIPOFECTAMINE™ 3000 brand transfection reagent according to the manufacturer's protocol (Thermo Fisher Scientific). 20 hours post-transfection, the cells were treated with 25 μM SuTEx probes for 2 hours in DMEM medium without FBS. The cells were then treated with 75 μM NaAsO₂ for 30 minutes and fixed with 3% paraformaldehyde in PBS for 15 minutes at RT and washed with PBS twice. The same protocols for IF of HeLa cells and quantification of G3BP1-positive SGs described above were followed thereafter.

Data Analysis—Chemical Proteomics. Identification and analysis of peptides and proteins using LC-MS was accomplished using bioinformatics software and quality control parameter protocols as previously described in Hahm et al., 2020.

Data Analysis—Microscopy. All slides were blinded for imaging. Leica SP5X images were analyzed using Fiji (ImageJ). Custom macros were written for analysis and can be obtained upon request. To elaborate, images were split into channels based on the different laser wavelengths measured. Image stacks were then combined using ‘Z-Project’ with an average intensity parameter and a threshold was applied to exclude noise based on the negative control (no EDC4 antibody). The ‘Analyze Particles’ function was used with relevant size and circularity constraints (0.85-1.00) to count the number of foci, with total PBs having a diameter of >150 nm, and large PBs>450 nm. The number of foci was normalized to the number of cells (defined by a DAPI stain count) in each linage, and the number of foci per cell was averaged across technical replicates. Every sample had at least two technical replicates and two independent biological replicates. Data were entered manually into a Microsoft Excel file. Inhibitors were normalized to a % vehicle and GraphPad PRISM (ver. 7) software was used to run statistical tests and generate plots.

Data Analysis—NMR. NMR spectra were analyzed and annotated using Mnova (Mestre lab Research, ver. 10.0.0) software.

Data Analysis—Statistics. GraphPad PRISM (ver. 7) software was used to perform statistical tests on the generated plots.

Data Analysis—Gene Ontology. For fold-enrichments, maximum values were set to 100 and minimum values to 0.01 before calculating the log 2-scaled value.

Lipophilic efficiency calculations for AHL-030 and HHS-166. Lipophilic efficiency was calculated as previously described in Leeson & Springthorpe, 2007 using IC₅₀ (AHL-030) and EC₅₀ (HHS-166) potency values from experiments and c Log P values from ChemDraw (Version 20.0.0.41 on a Windows PC):

$$LipE = p I {C}_{50} / {EC}_{50} - c Log P$$

| | AHL-030 | HHS-166 |
|---|---|---|
| IC₅₀/EC₅₀ (M) | 0.0000056 | 0.000008 |
| pIC₅₀/EC₅₀ | 5.3 | 5.1 |
| clogP | −1.8 | 2.5 |
| LipE | 7 | 3 |

Chemical Synthesis. All chemicals used were reagent grade and used as supplied, except where noted. Dichloromethane (DCM), chloroform, toluene, ethyl acetate (EtOAc), methanol (MeOH), hexanes, n-heptane, acetone, and acetonitrile (ACN) were used without any further purification steps. Analytical thin layer chromatography (TLC) was performed on Merck silica gel 60 F254 plates (0.25 mm). ¹H, ¹⁹F, and ¹³C NMR spectra were recorded on Varian Inova 500 (500 MHz), 600 (600 MHz), or Bruker Avance III 800 (800 MHz) spectrometers in CDCl₃, acetone-d₆, or DMSO-d₆ with chemical shifts referenced to internal standards (CDCl₃: 7.26 ppm ¹H, 77.16 ppm ¹³C; (CD₃)₂CO: 2.05 ppm ¹H, 29.84 and 206.26 ppm ¹³C; (CD₃)₂SO: 2.50 ppm ¹H, 40.00 ppm ¹³C), unless stated otherwise. Splitting patterns are indicated as s, singlet; d, doublet; t, triplet; q, quartet; m, multiplet; br, broad singlet for ¹H-NMR data. NMR chemical shifts (δ) are reported in ppm and coupling constants (J) are reported in Hz. High resolution mass spectral (HRMS) data were obtained by an Agilent 6545B LC/Q-TOF (Agilent Technologies, Santa Clara, California, United States of America). High-performance liquid chromatography (HPLC) data was obtained by a Shimadzu 1100 Series spectrometer with UV detection at 254 nm using a KINETEX® 2.6 μm C18 100 Å LC column (50×4.6 mm) with a 10-minute acidified water/acetonitrile gradient as previously described in Hahm et al., 2020.

Chemical Suppliers. The chemicals listed below were purchased commercially and were all >95% pure. Solvents purchased commercially were HPLC grade:
Enamine: 1H-1,2,3-Triazole
Fisher Scientific: Triethylamine, Hydrazine hydrate
Fisher Chemical: Water, Chloroform (LC-MS Grade), Toluene, MeOH (LC-MS Grade), ACN, DCM, Acetone, EtOAc, n-Heptane, Hexanes, Formic acid (OPTIMA®), Acetic acid (OPTIMA®), Sodium sulfate (anhyd.), Magnesium sulfate (anhyd.), Sodium carbonate (anhyd.), N,N-Dimethylformamide dimethyl acetal
Combi-Blocks: Isopropylsulfonyl chloride, 1-Naphthalenesulfonyl chloride, Copper(I) thiophene-2-carboxylate (‘CuTC’), 3-Chlorophenyl acetylene, 1-Ethynyl-2-fluorobenzene, 1-Bromo-2-ethynylbenzene, 1-Ethynyl-4-(trifluoromethyl)benzene, 1-Butyl-4-eth-1-ynylbenzene, 4-(Trifluoromethyl)benzamide
Acros Organics: 1,2,4-IH-Triazole, 4-Fluorobenzenesulfonyl chloride, 4-Bromobenzeuesulfonyl chloride, 4-Cyanobenzenesulfouyl chloride, 2-Naphthalenesulfonyl chloride, 4-Methoxyphenyl sulfonyl chloride, Pyridine-3-sulfonyl chloride, 4-Biphenylsulfonyl chloride, 1-Nonyne, Cyclohexylacetylene, Phenylacetylene
Oakwood Chemicals: 4-(Trifluoromethoxy)benzenesulfonyl chloride, Cyclopropanesulfonyl chloride, 2,4,6-Triisopropylbenzenesulfonyl azide solution (10%±2% in toluene), Nicotinamide
Sigma-Aldrich: Sodium azide (99%), 3-(Methylsulfonyl)-1-propyne, 1-Octyne, 2,4-Difluorophenylacetylene, 4-Methoxybenzoic acid
Alfa Aesar: 4-Acetamidobenzenesulfonyl chloride
Decon Laboratories: 200 proof ethanol
eNovation Chemicals LLC: Tosyl azide
AstraTech: 4-Phenoxybenzenesulfonyl chloride
AK Scientific: 4-Pentylphenylacetylene
ArkPharm: 4-Ethynyl-1,1′-biphenyl, 1-Bromo-4-ethynylbenzene
Accela: 4-Ethynylanisole

General synthetic protocol for sulfonyl-azides. Sulfonyl chloride (50 mmol, 1 eq.) was dissolved in acetone (50 mL) in a 250 mL Erlenmeyer flask followed by the addition of water (10 mL). The solution was cooled to 0° C. with an ice bath while stirring. Sodium azide (105 mmol, 2.1 eq.) was dissolved in water (20 mL) and then added to the reaction flask. The reaction was stirred and slowly warmed to room temperature. Reaction completion was monitored by TLC. Upon completion, the reaction solution was concentrated in vacuo and the product was extracted with EtOAc (2×100 mL). The organic layer was washed with water (2×50 mL), 5% sodium carbonate (2×50 mL), and water (2×50 mL) before being dried over magnesium sulfate and concentrated to obtain the product.

General synthetic protocol for 1,2,3-SuTEx compounds. To a solution of sulfonyl azide (5 mmol, 1 eq.) vigorously stirring in toluene (50 mL) at room temperature in a 250 mL Erlenmeyer flask was added the alkyne (5 mmol, 1 eq.) followed by addition of CuTC (0.5 mmol, 0.1 eq.). Reaction completion was monitored by TLC. Upon completion, a saturated ammonium chloride solution was added (30 mL) and the reaction was stirred for 15 minutes before the addition of brine (30 mL). The flask was rinsed with EtOAc (5 mL) and the product extracted with EtOAc (2×25 mL). The organic layer was washed with brine (25 mL) and the aqueous layer was back-extracted with EtOAc (1×25 mL); the combined organic layers were washed with brine (1×25 mL) and dried over anhydrous sodium sulfate. The organic layers were concentrated to yield the crude product. The crude product was then recrystallized with hot EtOAc and n-Heptane and the crystallized product was isolated using vacuum filtration and dried to yield the final product.

General synthetic protocol for 1,2,4-SuTEx compounds (JWB180, JWB137, JWB154, JWB119). Sulfonyl chloride (1.0 mmol, 1.0 eq.) was dissolved in anhydrous ethanol (1.9 mL) and the triazole (1.0 mmol, 1.0 eq.) and triethylamine (124 μL, 1.1 mmol, 1.1 eq.) were added and the reaction was stirred at room temperature for 2 hours or until the product was precipitated out of solution. Filtrate was removed and the product was isolated and washed several times with cold ethanol. The product was concentrated in vacuo. More information about the synthesis and characterization of precursors and products is described in Brulet et al., 2020.

### 4-((4-(3-Chlorophenyl)-1H-1,2,3-triazol-1-yl)sulfonyl)benzonitrile (EKT231)

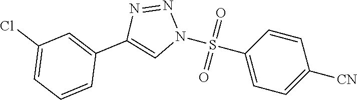 [SMILES: N#Cc1ccc(S(=O)(=O)n2cc(-c3cccc(Cl)c3)nn2)cc1]

As a white, fluffy solid, ¹H NMR (500 MHz, DMSO-d₆) δ 9.59 (s, ¹H), 8.36-8.34 (m, 2H), 8.25-8.24 (m, 2H), 8.01 (t, J=5 Hz, 1H), 7.93 (dt, J=10, 5 Hz, 1H), 7.53 (t, J=5 Hz, 1H), 7.50-7.48 (m, 1H). ¹³C NMR (201 MHz, DMSO-d₆) δ 146.28, 139.48, 134.96, 134.33, 131.54, 131.01, 129.48, 129.44, 126.07, 124.84, 123.25, 118.98, 117.51. ESI-TOF (HRMS) m/z [M+H]⁺ calculated for C15H10ClN4O₂S⁺: 345.0208. found: 345.0209.

### 4-(2,4-Difluorophenyl)-1-((4-(trifluo•·omethyl)phenyl)sulfonyl)-1H-1,2,3-triazole (EKT166)

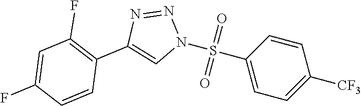 [SMILES: O=S(=O)(c1ccc(C(F)(F)F)cc1)n1cc(-c2ccc(F)cc2F)nn1]

As a white, fluffy solid, ¹H NMR (500 MHz, DMSO-d₆) δ 9.20 (d, J=3.5 Hz, 1H), 8.48-8.46 (m, 2H), 8.16-8.10 (m, 3H), 7.52-7.48 (m, 1H), 7.27 (td, J=10, 5 Hz, 1H). ¹³C NMR (201 MHz, DMSO-d₆) δ 163.09 (dd, J=249.12 Hz), 159.46 (dd, J=252.13 Hz), 140.84 (d, J=3 Hz), 139.52, 135.84 (q, J=32 Hz), 130.19-130.05 (m), 128.94, 128.14-128.08 (m), 125.44-121.36 (m), 123.87 (d, J=10 Hz), 113.68 (dd, J=13, 4 Hz), 113.02 (dd, J=22, 3 Hz), 105.38 (t, J=26 Hz). ¹⁹F NMR (564.32 MHz, CDCl₃) δ −63.58 (s, 3F), −107.74-−107.79 (m, IF), −109.59-−109.65 (m, 1F). ESI-TOF (HRMS) m/z [M+H]⁺ calculated for C₁₅H₉F₅N₃O₂S⁺: 390.0330. found: 390.0330.

### 1-((4-Methoxyphenyl)sulfonyl)-4-((methylsulfonyl)methyl)-1H-1,2,3-triazole (AHL-030)

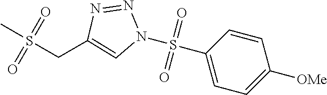 [SMILES: COc1ccc(S(=O)(=O)n2cc(CS(C)(=O)=O)nn2)cc1]

As a white, fluffy solid, ¹H NMR (600 MHz, DMSO-d6) o 8.82 (s, 1H), 8.10-8.08 (m, 2H), 7.24-7.21 (m, 2H), 4.65 (s, 2H), 3.86 (s, 3H), 3.01 (s, 3H). 13C NMR (201 MHz, DMSO-d6) δ 165.89, 137.30, 131.67, 126.07, 125.82, 116.26, 56.72, 50.85, 40.61. ESI-TOF (HRMS) m/z [M+H]⁺ calculated for CnH14N30sS2+: 332.0369. found: 332.0373.

### 4-Heptyl-1-((4-methoxyphenyl)sulfonyl)-1H-1,2,3-triazole (HHS-173)

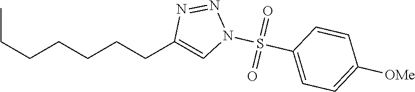 [SMILES: CCCCCCCc1cn(S(=O)(=O)c2ccc(OC)cc2)nn1]

As a white, fluffy solid, ¹H NMR (500 MHz, CDCl₃) δ 8.07-8.04 (m, 2H), 7.85 (s, 1H), 7.06-7.03 (m, 2H), 3.91 (s, 3H), 2.72 (t, J=10 Hz, 2H), 1.70-1.64 (m, 2H), 1.36-1.32 (m, 4H), 1.31 (s, 1H), 1.30-1.27 (m, 3H), 0.91-0.88 (m, 3H). ¹³C NMR (201 MHz, CDCl₃) δ 165.19, 148.25, 131.08, 127.32, 120.07, 114.98, 55.92, 31.69, 29.09, 28.94, 28.94, 25.42, 22.61, 14.07. ESI-TOF (HRMS) m/z [M+H]⁺ calculated for C₁₆H₂₄N₃O₃S⁺: 338.1533. found: 338.1535.

### 3-((4-(2-Fluorophenyl)-1H-1,2,3-triazol-1-yl)sulfonyl)pyridine (AMC-001)

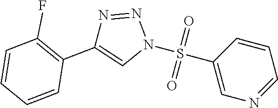 [SMILES: O=S(=O)(c1cccnc1)n1cc(-c2ccccc2F)nn1]

As a yellow solid, ¹H NMR (500 MHz, DMSO-d₆) δ 9.40 (d, J=2.5 Hz, 1H), 9.17 (d, J=2.5 Hz, 1H), 9.04 (dd, J=5, 2.5 Hz, 1H), 8.66 (dt, J=10, 5 Hz, 1H), 8.10 (td, J=5, 2.5 Hz, 1H), 7.81 (dd, J=5, 2.5 Hz, 1H), 7.54-7.49 (m, 1H), 7.44-7.35 (m, 2H). ¹³C NMR (201 MHz, DMSO-d₆) δ 159.83, 158.59, 157.02, 149.11, 141.35, 137.20, 132.86, 131.68 (d, J=8 Hz), 128.62 (d, J=2 Hz), 125.63 (m), 123.97 (d, J=12 Hz), 116.84 (d, J=12 Hz), 116.73 (d, J=20 Hz). ¹⁹F NMR (564.32 MHz, CDCl₃) δ −113.45-−113.50 (m, IF). ESI-TOF (HRMS) m/z [M+H]⁺ calculated for C₁₃H₁₀FN₄O₂S⁺: 305.0503. found: 305.0504.

### 4-(4-Butylphenyl)-1-(naphthalen-2-ylsulfonyl)-1H-1,2,3-triazole (AHL-012)

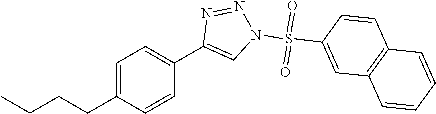 [SMILES: CCCCc1ccc(-c2cn(S(=O)(=O)c3ccc4ccccc4c3)nn2)cc1]

As a white, fluffy solid, ¹H NMR (600 MHz, CDCl₃) δ 8.76 (m, 1H), 8.32 (s, 1H), 8.03-7.99 (m, 3H), 7.91 (d, J=12 Hz, 1H), 7.72-7.69 (m, 3H), 7.67-7.64 (m, 1H), 7.23-7.21 (m, 2H), 2.62-2.60 (m, 2H), 1.61-1.56 (m, 2H), 1.37-1.30 (m, 2H), 0.90 (t, J=6 Hz, 3H). ¹³C NMR (201 MHz, DMSO-d₆) δ 147.56, 143.94, 136.04, 132.63, 131.98, 131.43, 131.32, 131.22, 130.46, 129.39, 128.98, 128.59, 126.51, 126.25, 122.17, 121.52, 35.02, 33.38, 22.15, 14.21. ESI-TOF (HRMS) m/z [M+H]⁺ calculated for C₂₂H₂₂N₃O₂S⁺: 392.1427. found: 392.1434.

### 4-(2-Fluorophenyl)-1-((4-phenoxyphenyl)sulfonyl)-1H-1,2,3-triazole (EKT100)

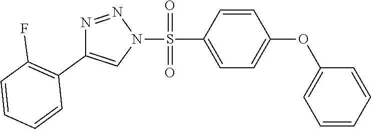 [SMILES: O=S(=O)(c1ccc(Oc2ccccc2)cc1)n1cc(-c2ccccc2F)nn1]

As a white solid, ¹H NMR (600 MHz, CDCl₃) δ 8.47 (d, J=6 Hz, 1H), 8.28-8.25 (m, 1H), 8.09-8.07 (m, 2H), 7.43-7.40 (m, 2H), 7.37-7.33 (m, 1H), 7.27-7.24 (m, 2H), 7.15 (dd, J=12, 6 Hz, 1H), 7.07-7.04 (m, 4H). ¹³C NMR (201 MHz, DMSO-d₆) δ 164.39, 159.81, 158.57, 154.17, 141.12, 132.04, 131.57 (d, J=8 Hz), 131.07, 128.61 (d, J=4 Hz), 128.36, 126.30, 125.60 (d, J=4 Hz), 123.56-123.50 (d, J=12 Hz), 121.24, 118.42, 116.99 (d, J=12 Hz), 116.71 (d, J=22 Hz). ¹⁹F NMR (564.32 MHz, CDCl₃) δ −113.61-−113.66 (m, 1F). ESI-TOF (HRMS) m/z [M+H]⁺ calculated for C₂₀H₁₅FN₃O₃S⁺: 396.0813. found 396.0817.

### 4-([1,1′-Biphenyl]-4-yl)-1-(cyclopropylsulfonyl)-1H-1,2,3-triazole (EKT235)

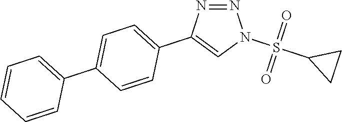 [SMILES: O=S(=O)(C1CC1)n1cc(-c2ccc(-c3ccccc3)cc2)nn1]

As a tan, crystalline solid, ¹H NMR (500 MHz, DMSO-d₆) δ 9.35 (s, 1H), 8.11-8.09 (m, 2H), 7.84-7.82 (m, 2H), 7.77-7.74 (m, 2H), 7.50 (t, J=10 Hz, 2H), 7.42-7.39 (m, 1H), 3.51-3.46 (m, 1H), 1.52-1.48 (m, 2H), 1.42-1.36 (m, 2H). ¹³C NMR (201 MHz, DMSO-d₆) δ 146.70, 140.97, 139.79, 129.49, 128.42, 128.26, 127.74, 127.10, 126.82, 122.17, 32.21, 8.08. ESI-TOF (HRMS) m/z [M+H]⁺ calculated for C₁₇H₁₆N₃O₂S⁺: 326.0958. found: 326.0959.

### 1-(Cyclopropylsulfonyl)-4-(3-fluorophenyl)-1H-1,2,3-triazole (EKT179)

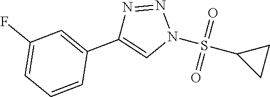 [SMILES: O=S(=O)(C1CC1)n1cc(-c2cccc(F)c2)nn1]

As a white, fluffy solid, ¹H NMR (500 MHz, DMSO-d₆) δ 9.39-9.37 (m, 1H), 7.87-7.80 (m, 2H), 7.58-7.54 (m, 1H), 7.29-7.24 (m, 1H), 3.49-3.44 (m, 1H), 1.49-1.46 (m, 2H), 1.40-1.38 (m, 2H). ¹³C NMR (201 MHz, DMSO-d₆) δ 163.50 (d, J=42 Hz), 145.87 (d, J=2 Hz), 137.78-137.74 (d, J=8 Hz), 131.70-131.65 (d, J=10 Hz), 122.93, 122.30-122.29 (d, J=2 Hz), 116.21 (d, J=20 Hz), 112.99 (d, J=18 Hz), 32.20, 8.12. ¹⁹F NMR (564.32 MHz, CDCl₃) δ −111.98 (q, J=7 Hz, 1F). ESI-TOF (HRMS) r/z [M+H]⁺ calculated for C₁₁H₁₁FN₃O₂S⁺: 268.0551. found: 268.0554.

### 4-(4-Trifluoromethyl)phenyl)-1-((2,4,6-triisopropylphenyl)sulfonyl)-1H-1,2,3-triazole (AHL-003)

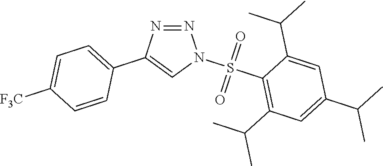 [SMILES: CC(C)c1cc(C(C)C)c(S(=O)(=O)n2cc(-c3ccc(C(F)(F)F)cc3)nn2)c(C(C)C)c1]

As a white, fluffy solid, ¹H NMR (600 MHz, DMSO-d₆) δ 9.74 (s, 1H), 8.22 (d, J=6 Hz, 2H), 7.87 (d, J=6 Hz, 2H), 7.45 (s, 2H), 4.13-4.07 (m, 2H), 3.03-2.98 (m, 1H), 1.26-1.22 (m, 8H), 1.16 (d, J=6 Hz, 10H). ¹³C NMR (201 MHz, DMSO-d₆) δ 157.23, 152.63, 150.77, 145.65, 133.27, 129.61, 129.45, 128.60, 126.95, 126.53 (t, J=3 Hz), 125.50, 124.89, 122.35, 34.07, 34.01, 29.98, 29.95, 25.30, 24.86, 24.59, 23.69, 23.58. ¹⁹F NMR (564.32 MHZ, CDCl₃) δ −62.78 (s, 3F). ESI-TOF (HRMS) m/z [M+H]⁺ calculated for C₂₄H₂₉F₃N₃O₂S⁺: 480.1927. found: 480.1931.

### 1-4Naphthalen-1-ylsulfonyl)-4-(4-pentylphenyl)-1H-1,2,3-triazole (AHL-016)

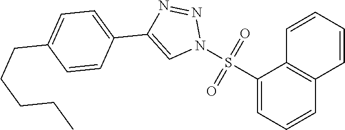 [SMILES: CCCCCc1ccc(-c2cn(S(=O)(=O)c3cccc4ccccc34)nn2)cc1]

As a fluffy, white solid, ¹H NMR (600 MHz, CDCl₃) δ 8.86-8.84 (m, 1H), 8.63-8.62 (m, 1H), 8.36 (s, 1H), 8.22-8.20 (d, J=12 Hz, 1H), 7.95-7.93 (d, J=12 Hz, 1H), 7.73-7.63 (m, 4H), 7.63-7.60 (m, 1H), 7.22-7.20 (m, 2H), 2.61-2.58 (t, J=6 Hz, 2H), 1.62-1.57 (m, 2H), 1.34-1.26 (m, 4H), 0.88-0.85 (t, J=6 Hz, 3H). ¹³C NMR (201 MHz, DMSO-d₆) δ 147.35, 143.97, 138.72, 134.26, 133.14, 130.40, 130.36, 130.29, 129.38, 128.33, 127.53, 126.45, 126.21, 125.56, 123.25, 121.59, 35.29, 31.26, 30.88, 22.37, 14.35. ESI-TOF (HRMS) m/z [M+H]⁺ calculated for C₂₃H₂₄N₃O₂S⁺: 406.1584. found: 406.1585.

### 1-([1,1′-Biphenyl]-4-ylsulfonyl)-4-(2-bromophenyl)-1H-1,2,3-tl'iazole (EKT132)

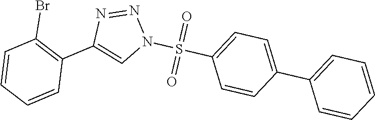 [SMILES: O=S(=O)(c1ccc(-c2ccccc2)cc1)n1cc(-c2ccccc2Br)nn1]

As a tan solid, ¹H NMR (600 MHz, CDCl₃) δ 8.83 (s, 1H), 8.22-8.20 (m, 2H), 8.09 (dd, J=6, 3 Hz, 1H), 7.81-7.78 (m, 2H), 7.66 (dd, J=6, 3 Hz, 1H), 7.61-7.57 (m, 2H), 7.49-7.46 (m, 2H), 7.45-7.42 (m, 1H), 7.42-7.39 (m, 1H), 7.24-7.22 (m, 1H). ¹³C NMR (201 MHz, DMSO-d₆) δ 148.26, 145.60, 138.05, 134.08, 133.99, 131.70, 131.49, 129.88, 129.82, 129.73, 129.60, 129.04, 128.60, 127.91, 124.42, 121.93. ESI-TOF (HRMS) m/z [M+H]⁺ calculated for C₂₀H₁₅BrN₃O₂S⁺: 440.0063. found: 440.0068.

### 3-(1-((4-Fluorophenyl)sulfonyl)-1H-1,2,4-triazol-3-yl)pyridine (JWB180)

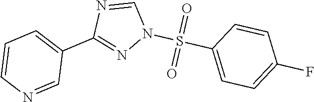 [SMILES: O=S(=O)(c1ccc(F)cc1)n1cnc(-c2cccnc2)n1]

As a white, fluffy solid, ¹H NMR (800 MHz, DMSO-d₆) δ 9.63 (s, 1H), 9.24 (m, 1H), 8.78 (dd, J=8, 4 Hz, 1H), 8.43-8.41 (m, 1H), 8.36-8.34 (m, 2H), 7.71-7.66 (m, 2H), 7.65-7.63 (m, 1H). ¹³C NMR (201 MHz, DMSO-d₆) δ 167.38, 166.11, 162.44, 151.77, 148.09 (d, J=173 Hz), 134.84, 132.48 (d, J=20 Hz), 131.75 (d, J=2 Hz), 125.28, 124.73, 118.8 (d, J=24 Hz). ¹⁹F NMR (564.32 MHz, CDCl₃) δ −98.86-−98.90 (m, 1F). ESI-TOF (HRMS) m/z [M+H]⁺ calculated for C₁₃H₁₀FN₄O₂S⁺: 305.0503. found: 305.0502.

### N-(4-((4-Phenyl-1H-1,2,3-triazol-1-yl)sulfonyl)phenylacetamide (AHL-005)

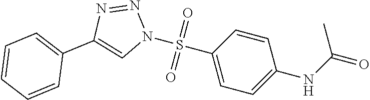 [SMILES: CC(=O)Nc1ccc(S(=O)(=O)n2cc(-c3ccccc3)nn2)cc1]

As a white solid, ¹H NMR (600 MHz, DMSO-d₆) δ 10.57 (s, 1H), 9.32 (s, 1H), 8.08-8.07 (m, 2H), 7.90-7.89 (m, 2H), 7.86-7.85 (m, 2H), 7.45-7.43 (m, 2H), 7.38-7.35 (m, 1H), 2.06 (s, 3H). ¹³C NMR (201 MHz, DMSO-d₆) δ 170.01, 147.30, 146.66, 130.50, 129.50, 129.48, 129.18, 128.16, 126.25, 121.64, 119.67, 24.72. ESI-TOF (HRMS) r/z [M+H]⁺ calculated for C₁₆H₁₅N₄O₃S⁺: 343.0859. found: 343.0860.

### 4-(3-(4-Methoxyphenyl)-1H-1,2,4-triazol-1-yl)sulfonyl)benzonitrile (JWB137)

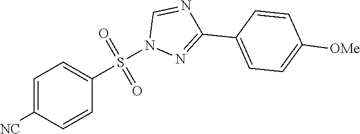

JWB137 was synthesized and characterized as described in Brulet et al., 2020.

### 4-Cyclohexyl-1-((4-methoxyphenyl)sulfonyl)-111-1,2,3-triazole (HHS-166)

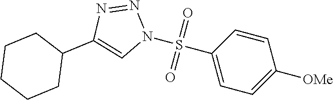 [SMILES: COc1ccc(S(=O)(=O)n2cc(C3CCCCC3)nn2)cc1]

As a white solid, ¹H NMR (500 MHz, CDCl₃) δ 8.06-8.04 (m, 2H), 7.81 (s, 1H), 7.05-7.02 (m, 2H), 3.89 (s, 3H), 2.79-2.74 (m, 1H), 2.08-2.03 (m, 2H), 1.82-1.79 (m, 2H), 1.75-1.71 (m, 1H), 1.44-1.34 (m, 4H), 1.29-1.23 (m, 1H). ¹³C NMR (201 MHz, DMSO-d₆) δ 165.52, 153.34, 131.28, 126.84, 121.11, 116.09, 56.65, 34.76, 32.41, 25.89, 25.86. ESI-TOF (HRMS) m/z [M+H]⁺ calculated for C₁₅H₂₀N₃O₃S: 322.1220. found: 322.1221.

### 4-(2,4-Difluorophenyl)-1-(isopropylsulfonyl)-1H-1,2,3-triazole (EKT158)

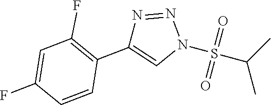 [SMILES: CC(C)S(=O)(=O)n1cc(-c2ccc(F)cc2F)nn1]

As a slight blue solid, ¹H NMR (500 MHz, DMSO-d₆) δ 8.91 (d, J=5 Hz, 1H), 8.18 (td, J=15, 10 Hz, 1H), 7.52-7.48 (m, 1H), 7.31-7.27 (m, 1H), 4.28-4.20 (m, 1H), 1.33 (d, J=10 Hz, 6H). ¹³C NMR (201 MHz, DMSO-d₆) δ 163.01 (dd, J=261, 12 Hz), 159.49 (dd, J=265, 12, 12 Hz), 158.69 (d, J=14 Hz), 140.25 (d, J=4 Hz), 130.14-130.07 (m), 124.63 (d, J=10 Hz), 113.98 (dd, J=16, 2 Hz), 113.00 (dd, J=26.4 Hz), 105.36 (t, J=26 Hz), 57.43, 16.01. ¹⁹F NMR (564.32 MHz, CDCl₃) δ −108.12-108.17 (m, 1F), −109.65-−109.71 (m, 1F). ESI-TOF (HRMS) m/z [M+H]⁺ calculated for C₁₁H₁₂F₂N₃O₂S⁺: 288.0613. found: 288.0616.

### 1-((4-(Trifluoromethoxy)phenyl)sulfonyl)-3-(4-(trifluoromethyl)phenyl)-1H-1,2,4-triazole (JWB154)

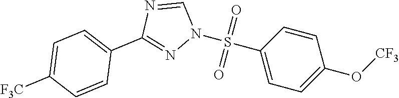 [SMILES: O=S(=O)(c1ccc(OC(F)(F)F)cc1)n1cnc(-c2ccc(C(F)(F)F)cc2)n1]

As a white, fluffy solid, ¹H NMR (500 MHz, DMSO-d₆) δ 9.58 (s, 1H), 8.35-8.32 (m, 2H), 8.22-8.20 (m, 2H), 7.89-7.87 (m, 2H), 7.75-7.72 (m, 2H). ¹³C NMR (201 MHz, DMSO-d₆) δ 163.36, 153.76, 148.73, 134.07, 132.85, 131.94, 131.15 (d, J=32 Hz), 127.85, 126.54 (d, J=4 Hz), 124.39 (d, J=271 Hz), 122.53, 120.16 (d, J=259 Hz). ¹⁹F NMR (564.32 MHz, CDCl₃) δ −57.70 (s, 3F), −62.97 (s, 3F). ESI-TOF (HRMS) m/z [M+H]⁺ calculated for C₁₆H₁₀F₆N₃O₃S⁺: 438.0342. found: 438.0340.

### 4-Hexyl-1-tosyl-1H-1,2,3-triazole (AHL-006)

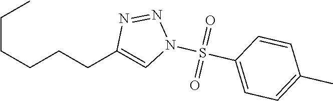 [SMILES: CCCCCCc1cn(S(=O)(=O)c2ccc(C)cc2)nn1]

As a white semisolid, ¹H NMR (500 MHz, CDCl₃) δ 8.00-7.98 (m, 2H), 7.86 (s, 1H), 7.40-7.38 (m, 2H), 2.73-2.70 (m, 2H), 2.46 (s, 3H), 1.69-1.63 (m, 2H), 1.37-1.28 (m, 6H), 0.90-0.87 (m, 3H). ¹³C NMR (201 MHz, CDCl₃) δ 148.36, 147.04, 133.35, 130.36, 128.55, 120.24, 31.45, 28.87, 28.77, 25.40, 22.49, 21.82, 14.02. ESI-TOF (HRMS) m/z [M+H]⁺ calculated for C₁₅H₂₂N₃O₂S⁺: 308.1427. found: 308.1430.

### 3-(4-Bromophenyl)-1-((4-bromophenylsulfonyl)-1H-1,2,4-triazole (JWB119)

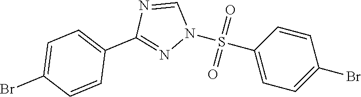 [SMILES: O=S(=O)(c1ccc(Br)cc1)n1cnc(-c2ccc(Br)cc2)n1]

As a white, fluffy solid, ¹H NMR (800 MHz, DMSO-d₆) δ 9.51 (s, 1H), 8.09-8.07 (m, 2H), 7.99-7.95 (m, 2H), 7.94-7.92 (m, 2H), 7.73-7.71 (m, 2H). ¹³C NMR (201 MHz, DMSO-d₆) δ 148.13, 146.63, 132.36, 131.08, 129.40, 128.40, 128.22, 123.23, 122.06, 99.99. ESI-TOF (HRMS) m/z [M+H]⁺ calculated for C₁₄H₁₀Br₂N₃O₂S⁺: 441.8855. found: 441.8853.

### Example 1

### Phenotypic Screening for RNP-Granule-Modulating SuTEx Compounds

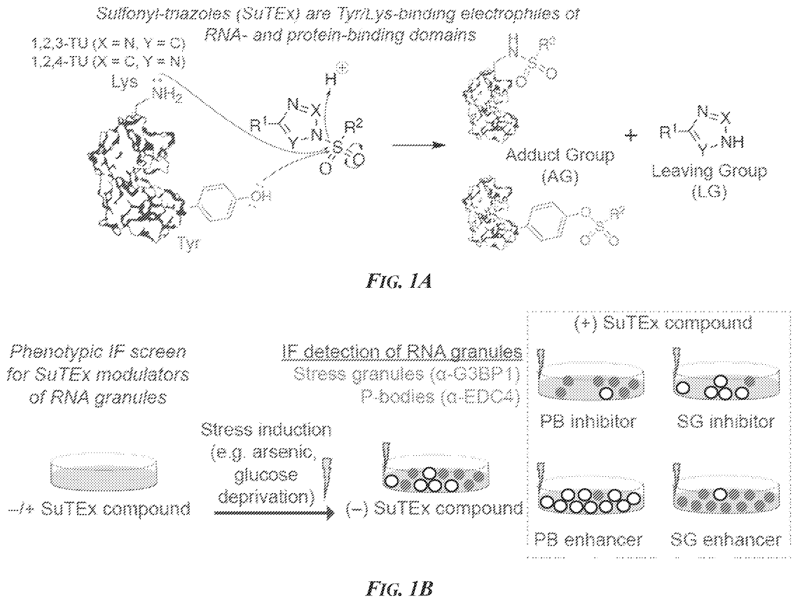

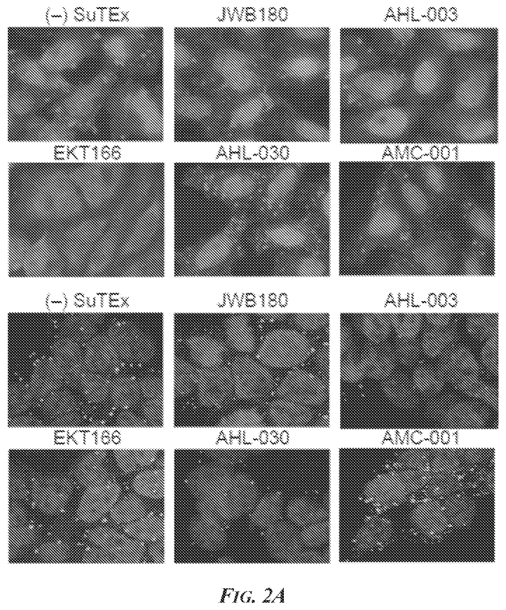

We reasoned screening of tyrosine (Tyr)- and lysine (Lys)-reactive electrophiles would facilitate discovery of RNP granule modulators due to the prevalence of these residues in protein-RNA interfaces (PRI; 2 and 6 residues/PRI, respectively; Kruger et al., 2018). Cysteines (Cys), by contrast, are found at a much lower frequency in PRIs (<1 residue/PRI), which further supports exploration of Tyr/Lys- vs Cys-targeting for the initial electrophile screen. Although sulfonyl-fluorides (SuFEx; Dong et al., 2014; Jones & Kelly, 2020) and -triazoles (SuTEx) both function as Tyr/Lys-binding electrophiles, we chose the latter because of capabilities for tuning reactivity and affinity though leaving group (LG) modifications (Grams & Hsu, 2022; FIGS. 1A and 7). Importantly, SuTEx probe binding activity in cells identified enrichment for RBD and protein-protein interaction (PPI) domains that are commonly found on RNP granule proteins (e.g., RRM and SH3 domains, respectively; Hubstenberger et al., 2017; Markmiller et al., 2018; Youn et al., 2019; Hahm et al., 2020). HEK293T and HeLa cells were chosen for screening and downstream analyses because these cell lines have served as model systems for cell biological and proteomic evaluation of PBs and SGs (Hubstenberger et al., 2017; Hallacli et al., 2022; Ciancone et al., 2022; Cui et al., 2023).

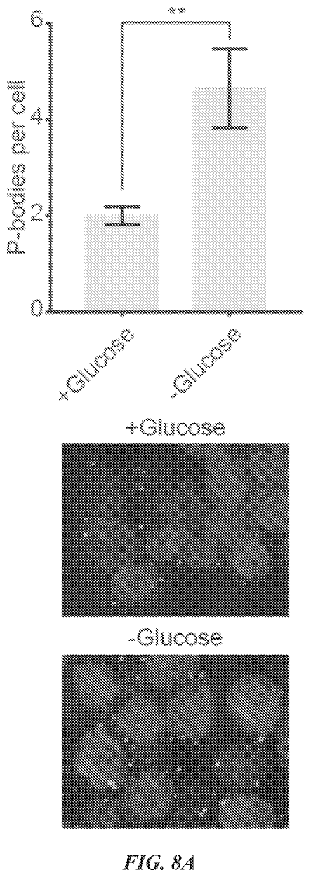

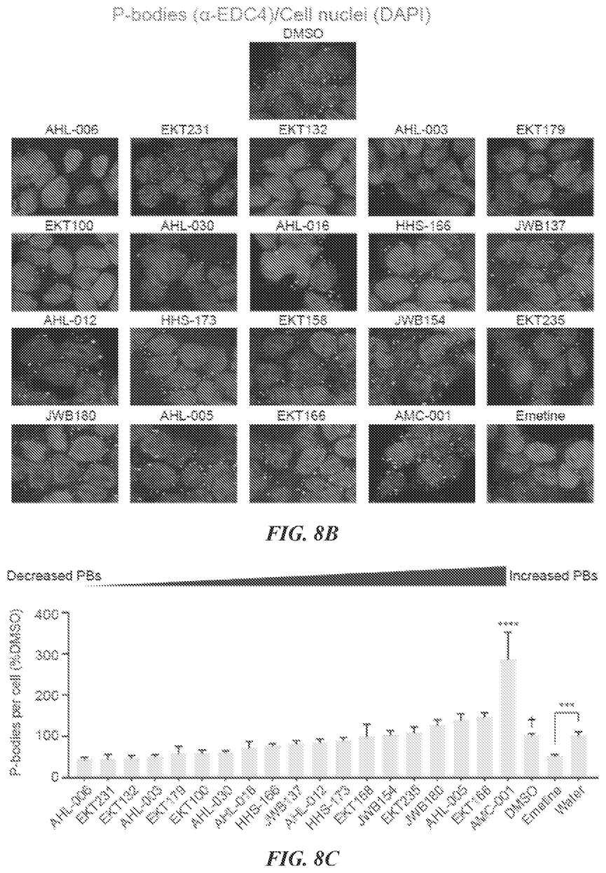

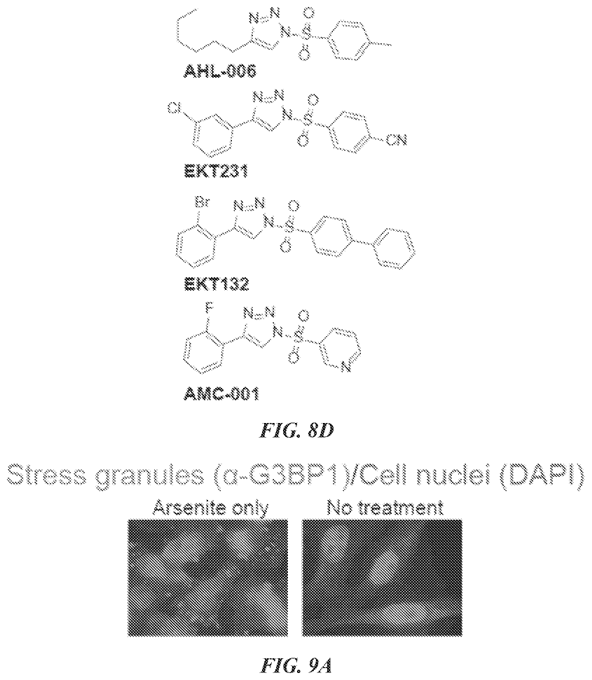

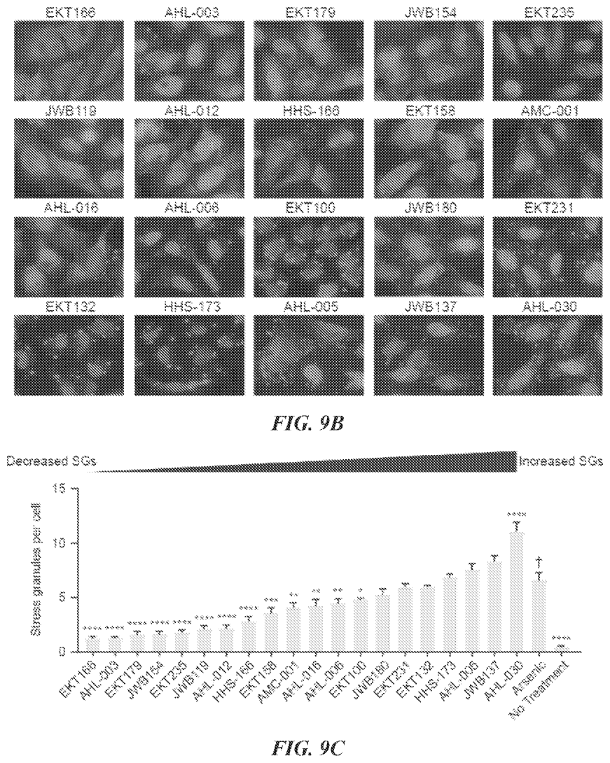

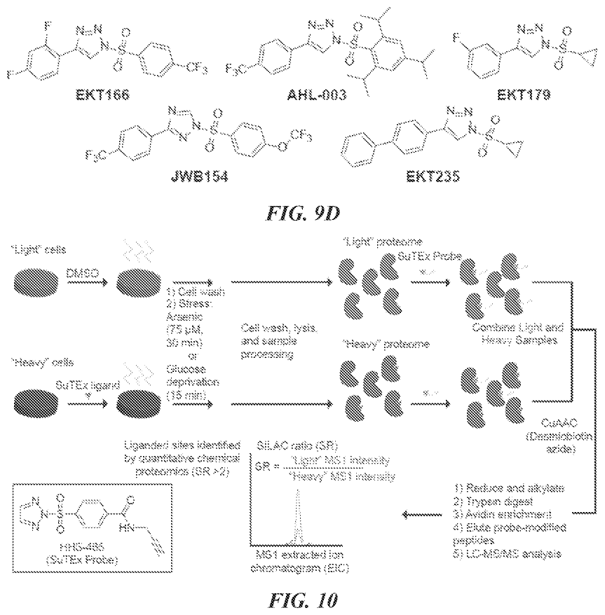

We used immunofluorescence (IF) detection for phenotypic screening of SuTEx compound activity on stress-induced PB and SG levels in cells (FIG. 1B). PBs were induced through glucose deprivation of HEK293T cells and detected by immunofluorescence with anti-enhancer of mRNA-decapping protein 4 (EDC4) as previously described (Kedersha & Anderson, 2007; Ciancone et al., 2022) (~4 vs 2 PBs/cell in −glucose and +glucose conditions, respectively; FIG. 8). EDC4 is an established PB marker for mammalian cells and is important for PB formation (Kedersha & Anderson, 2007; Luo et al., 2018). SGs were induced in HeLa cells by arsenite treatments and detected by fluorescent staining with anti-Ras GTPase-activating protein-binding protein 1 (G3BP1) antibody (75 μM arsenite, 30 min; FIG. 9). G3BP1 foci have been used in previous IF studies to identify SGs in cells (Kedersha & Anderson, 2007). See Materials and Methods for the EXAMPLES above for additional details of the PB and SG phenotypic screening workflow and data analysis.

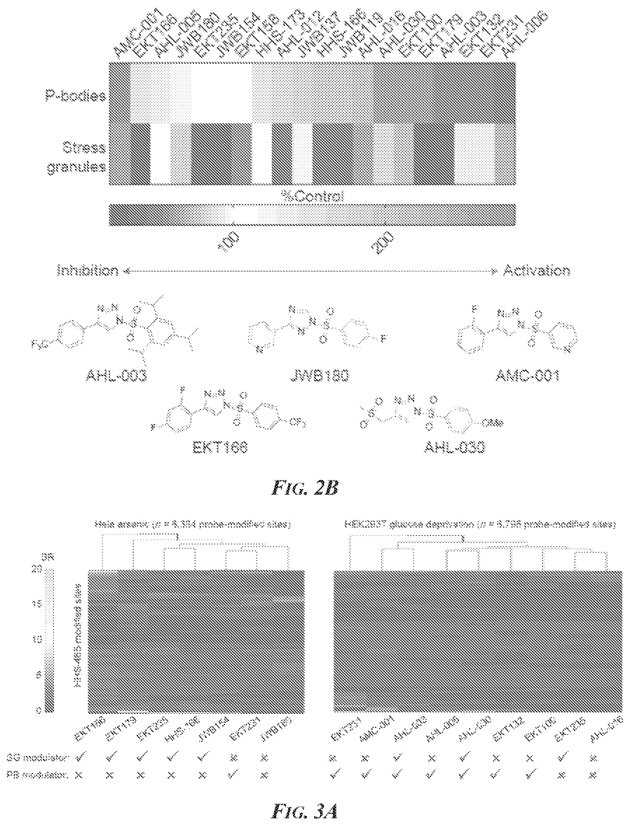

SuTEx compounds were selected for phenotypic screening based on fragment-like size (median MW of 343 g/mol), physicochemical properties (hydrophobicity, polar surface area, prevalence of sp³ centers) and LG diversity (1,2,3- and 1,2,4-triazoles; FIG. 7). We also included SuTEx fragments with alkyl substituents (e.g., EKT158, AHL-006 and HHS-166), which can temper reactivity and improve stability (Brulet et al., 2020). We identified several compounds that reproducibly decreased the number of PBs per cell in compound treated, glucose-deprived cells (>50% reduction in PBs/cell, 25 μM SuTEx fragment, 2 h; FIGS. 2 and 8). These PB inhibitors were enriched for 1,2,3-sulfonyl-triazoles containing alkyl- and aryl-substituents on both the adduct- and leaving-group (FIGS. 2 and 8). Several of these compounds showed a similar magnitude of PB blockade as the control compound and general protein translation inhibitor emetine (Kedersha & Anderson, 2007; Colombrita et al., 2009) (AHL-006, EKT231, and EKT132 vs emetine (50 nM, 2 h); FIG. 8C). Intriguingly, treatment of cells with AMC-001 resulted in a statistically significant increase in PBs per glucose-deprived cell (>2-fold increase in PBs/cells with AMC-001 pretreatment, FIGS. 2, 8B, and 8C).

Next, we evaluated SuTEx compound effects on cellular SG levels to determine if this class of electrophiles can modulate different types of RNP granules (FIG. 9). Several of the tested SuTEx compounds reduced cellular SG levels by >70% (EKT166, AHL-003) while other compounds displayed moderate, but statistically significant inhibitory activity (e.g., ~50% inhibition of SGs/cell by HHS-166, FIGS. 2 and 9B-9D). When compared with PB modulators, we detected compounds that could block both types of RNP granules (AHL-003) as well as SuTEx ligands with enriched activity for PBs (EKT132) and SGs (EKT166, HHS-166; FIGS. 2, 8, and 9). We also identified AHL-030 as an SG enhancer; treatment of cells with AHL-030 resulted in a ~2-fold increase in SGs per arsenite-treated cell (FIGS. 2 and 9C). Quantification and representative images from IF studies can be found in FIGS. 8 and 9.

In summary, our findings establish SuTEx ligands as a new class of electrophiles that can modulate cellular RNP granules in response to stress. The identification of distinct compounds for inhibiting or enhancing PBs and SGs in stressed cells should prove useful for functional investigation of these dynamic structures.

### Example 2

### Features of Protein Sites Liganded by SuTEx RNP Granule Modulators

An advantage of using covalent small molecules for ligand discovery is rapid target and binding site identifications using quantitative chemical proteomics. Target deconvolution represents an important, but often challenging, first step towards understanding mode of action for condensate-modifying compounds (Mitrea et al., 2022; Patel et al., 2022). The binding profiles obtained from chemical proteomics enable global selectivity profiling and bioinformatics-mediated discovery of enriched protein functions and domains underlying the PB- and SG-modulating activity of hit compounds.

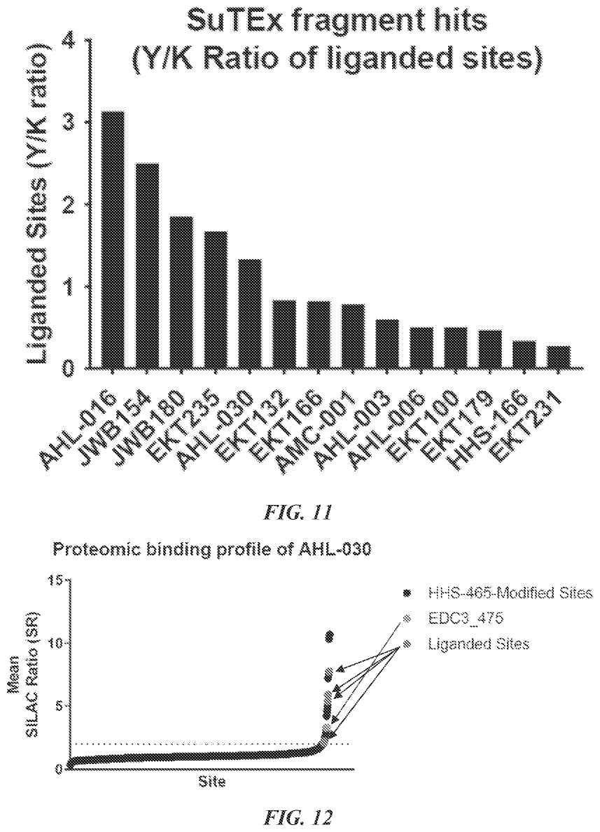

We performed quantitative liquid-chromatography tandem mass spectrometry (LC-MS/MS) chemical proteomic studies to identify the target protein and binding site(s) of SuTEx fragments with RNP granule modulating activity. Inactive SuTEx compounds were also included as negative controls for direct comparison. SILAC light and heavy cells (HEK293T and HeLa) were treated with experimental conditions used for phenotypic screening including the SuTEx ligand pretreatment (25 μM, 2 h) followed by PB (glucose-deprivation, 15 min) and SG induction (75 μM arsenite, 30 min). Afterwards, cells were lysed, soluble proteomes treated with HHS-465 SuTEx probe (100 μM, 1 h), a global Tyr/Lys-reactive probe used previously for RNP granule investigations (Ciancone et al., 2022), and samples processed to probe-modified peptides for LC-MS/MS analysis as previously reported (Huang et al., 2021), shown in FIG. 10.

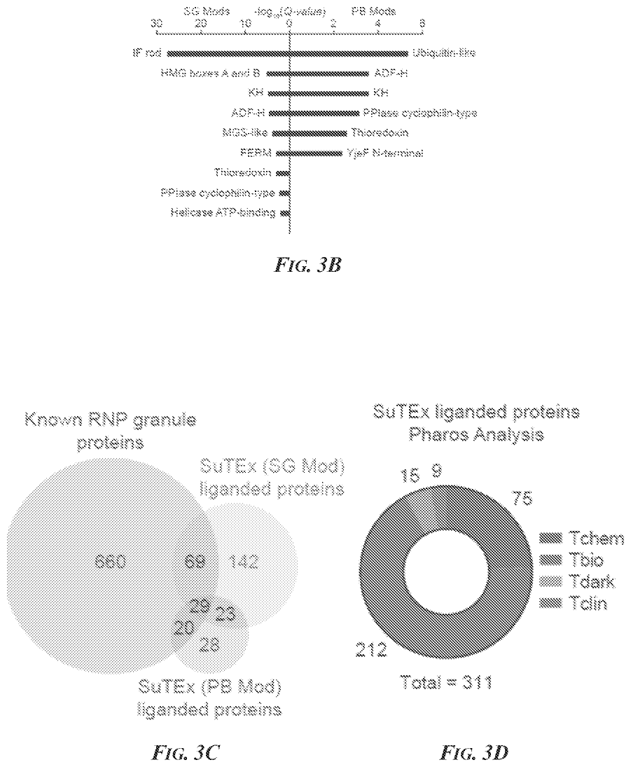

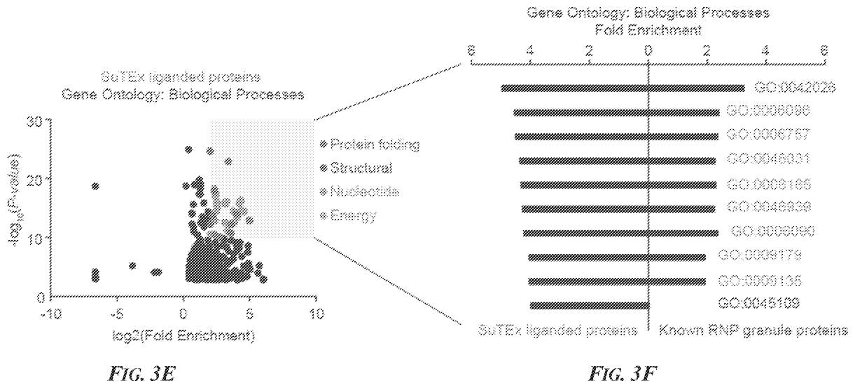

We detected ~8,700 probe-modified sites (Tyr and Lys) in our aggregate HeLa and HEK293T chemical proteomic analyses. Organization of SG and PB modulators by hierarchical clustering of SILAC ratios (SR) of detected Tyr and Lys sites from SuTEx ligand competition of probe labeling revealed evidence of grouping based on active vs inactive hits for each respective RNP granule type (FIG. 3A). We identified reproducibly liganded Tyr and Lys sites using the following criteria: 1) an average SILAC ratio (SR)>2 across biological replicates, and 2) a SR>2 in at least two biologically independent replicates. Using these criteria, a collection of 598 and 203 liganded sites (SR>2) from compound-treated, stress-induced HeLa and HEK293T cells, respectively, emerged for further bioinformatic analysis. SuTEx electrophile reactivity in proteomes was comparable to hit rates (i.e., fragment-competed residues/total sites quantified) from ABPP screens of cysteine-directed electrophile libraries (~3-9% for SuTEx compared with ~4-7% for cysteine-directed electrophiles; Backus et al., 2016; Kuljanin et al., 2021). Interestingly, the median Tyr/Lys ratio for all liganded sites was ~0.8, which supports a moderate preference for lysine binding of SuTEx ligand hits (FIG. 11).

Domain enrichment analyses of liganded sites identified statistically significant binding of SuTEx compounds at RBDs (KH, Helicase ATP-binding), ubiquitin-like, and YjeF N-terminal domains, which are reported to be involved in RNP granule regulation or liquid-liquid phase separation (UBL (Zheng et al., 2021), YjeF N-terminal (Ciancone et al., 2022), KH domains (Nielsen et al., 2002); FIG. 3B). Comparison of liganded proteins (311 proteins) to annotated RNP granule proteins (Hubstenberger et al., 2017; Youn et al., 2019) revealed substantial overlap (118 proteins, ~38% overlap; FIG. 3C). The remaining proteins without prior RNP granule annotation were enriched for functions involving cytoskeletal structures including intermediate filament proteins associated with the stress response and SG regulation (Toivola et al., 2010; Pattabiraman et al., 2020). A comparison of liganded proteins against the Pharos database (Sheils et al., 2021) showed differing levels of functional annotation and pharmacological tractability (FIG. 3D). Gene Ontology (GO) analysis of liganded proteins identified protein folding along with structural, nucleotide, and energetic cellular processes as enriched functions that were also prominently observed in GO analyses of the RNP granule proteome (FIGS. 3E and 3F).

A more detailed evaluation of liganded RNP granule proteins (Hubstenberger et al., 2017; Youn et al., 2019) identified SuTEx compounds targeting key protein families including chaperones (HSPB1, STIP1, calreticulin), nucleases (SND1, XRN2) and RNA-binding proteins (RBPs; PCBP1/2, HNRPK, hnRNPA/B). Several of these target proteins have demonstrated roles in phase separation (hnRNPA; Banani et al., 2017) or maintenance of the liquid state of phase-separated droplets (e.g., HSPB1 maintenance of phase-separated, cytoplasmic TDP-43 droplets; Lu et al., 2022). The liganded sites mapped to expected protein regions involved in RNP granule biology including RBDs (Y197 and K369 in the helicase domain of IF4A1; K23 in the KH domain of PCBP1) but also included domains mediating carbohydrate recognition (Y109 in the TNase-like domain of SND1) and dimerization (Y39 in the Phosphagen kinase N-terminal domain on KCRB). Many of the liganded residues are sites for post-translational regulation including phosphorylation (IF4A1-Y197, SND1-Y109, KCRB-Y39) and ubiquitination/sumoylation (PCBP1-K23, IF4A1-K369, HSPB1-K123, PARK7-K130) as annotated by PhosphoSitePlus (HTP>10 or LTP>3 cutoffs). Importantly, we found ~12% of RNP granule proteins liganded by SuTEx SG/PB modulators were not targeted by cysteine-reactive fragment electrophiles evaluated in large-scale, cell-based screens (Kuljanin et al., 2021).

### Example 3

### AHL-030 Covalently Binds the Stress Responsive EDC3 Y475 Site

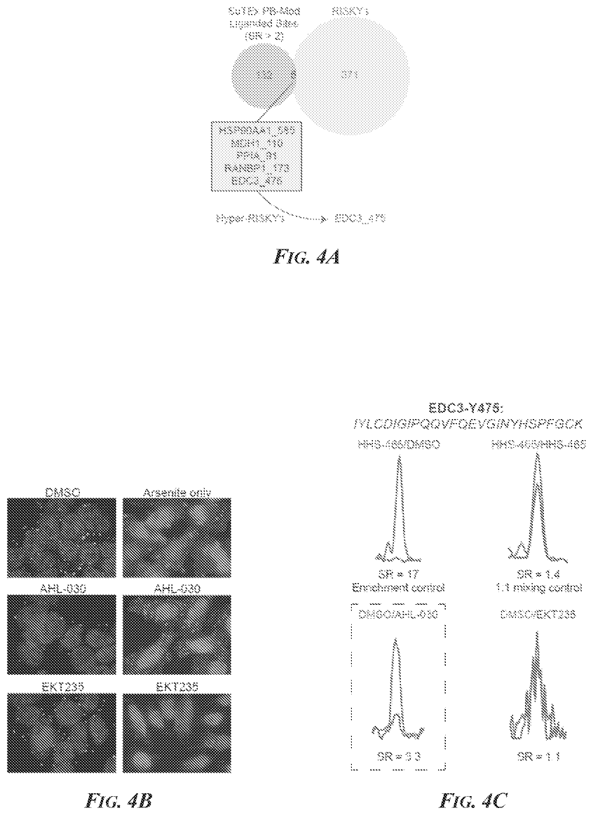

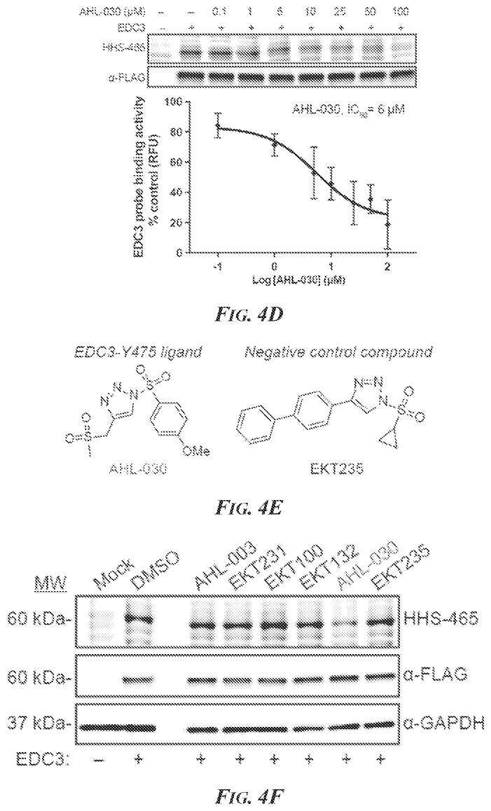

Next, we compared the collection of liganded sites with Tyr and Lys residues previously reported to couple stress response to RNP granule formation (i.e., RISKY sites (Ciancone et al., 2022); FIG. 4A). We reasoned this comparison would facilitate prioritization of sites with prior annotation in the stress response of cells. From this list we identified a set of liganded RISKY sites that included the hyper-reactive tyrosine (Y475) on EDC3 that is a component of PBs involved in removal of the 7MG 5′ mRNA cap (Hubstenberger et al., 2017; Luo et al., 2018; Ivanov et al., 2019). The Y475 site is located in the YjeF_N domain, which has been reported to function in EDC3 self-dimerization (Ling et al., 2008) and recently annotated as a arsenite-sensitive site that regulates PB formation through regulation of EDC3 phosphorylation state and PPIs (Ciancone et al., 2022).

Among the candidate PB-modulating SuTEx compounds, we focused on AHL-030 because of its PB-inhibitory, SG-enhancing activity in stressed cells and ability to ligand the EDC3 Y475 site (SR >2; FIGS. 4B and 4C). Importantly, we identified a restricted number of reproducibly liganded sites (7 in total) in addition to EDC3 Y475 in chemoproteomic profiling studies of AHL-030 (FIG. 12). We further confirmed AHL-030 as an EDC3 ligand using competitive activity-based protein profiling (ABPP; Grams & Hsu, 2022). Recombinant EDC3-expressing HEK293T cells were treated with a panel of RNP granule modulators including AHL-030 and the negative control compound EKT235 to evaluate structure-activity relationships (SAR, 25 μM compounds, 2 h). Cells were lysed and soluble proteomes labeled with HHS-465 (100 μM, 1 h, RT) followed by CuAAC with rhodamine-azide, SDS-PAGE and in-gel fluorescence scanning. By gel-based ABPP, we found that AHL-030 blocked HHS-465 probe labeling of EDC3 in a concentration-dependent manner as determined by reductions in fluorescent labeling of recombinant protein (IC₅₀=6 μM, FIGS. 4D and 4E). Using the in vitro IC₅₀, we calculated the lipophilic efficiency (LipE) of AHL-030 to be ~7, which falls in the range of acceptable lipophilicity in relation to potency (Leeson & Springthorpe, 2007). We showed the control compound (EKT235), additional PB (EKT231) and PB/SG modulators (AHL-003) were largely inactive against recombinant EDC3, which supports AHL-030 as a lead compound for future development of potent and selective EDC3-targeted ligands (FIG. 4F).

### Example 4

### Covalent Binding of G3BP1 Y40 Mediates the SG-Modulating Activity of HHS-166

RNP granule assembly and dissolution can be regulated through post-translational modifications (PTMs; Hofweber & Dormann, 2019). Protein phosphorylation, for example, regulates condensate formation through rapid and reversible modification of protein function, localization and interactions (Monahan et al., 2017; Tsang et al., 2019; Sridharan et al., 2022). Reported examples include phosphorylation of the RNA-binding protein fused in sarcoma (FUS) and fragile X mental retardation protein (FMRP), which results in reduced (Monahan et al., 2017) and increased condensate formation (Tsang et al., 2019), respectively. We compared liganded sites from reported SG proteins with assigned PTMs from PhosphoSitePlus (HTP score ≥10 or LTP score ≥1). We surmised this comparison would identify PTM sites that are amenable for developing targeted condensate-modulating compounds.

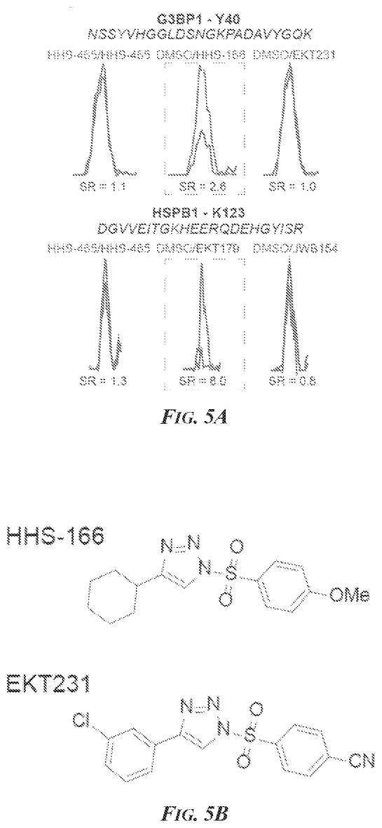

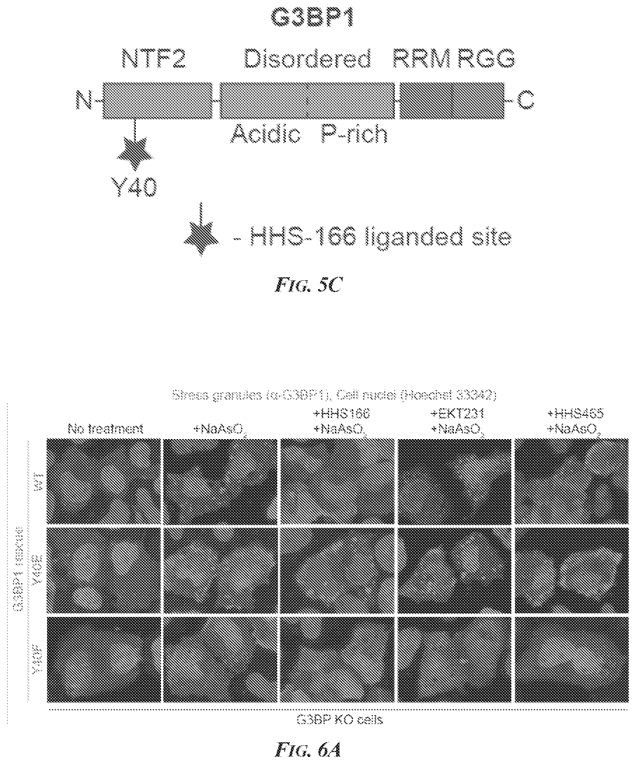

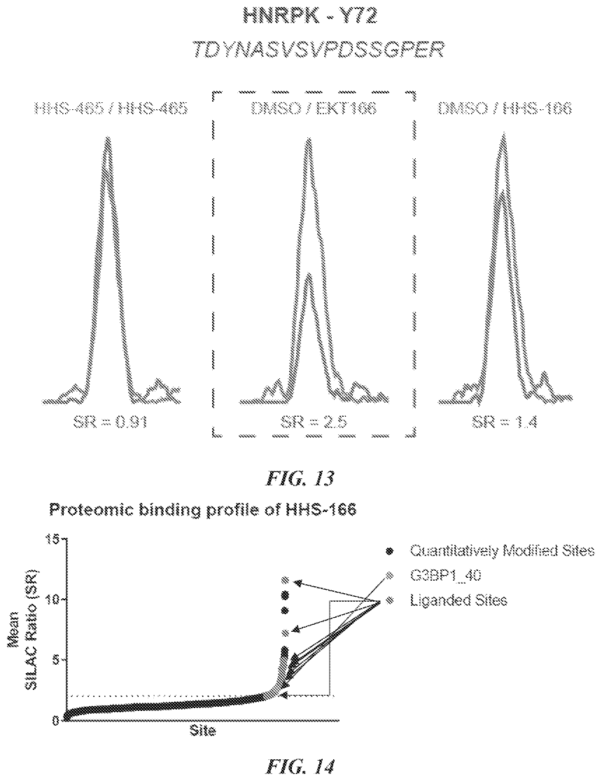

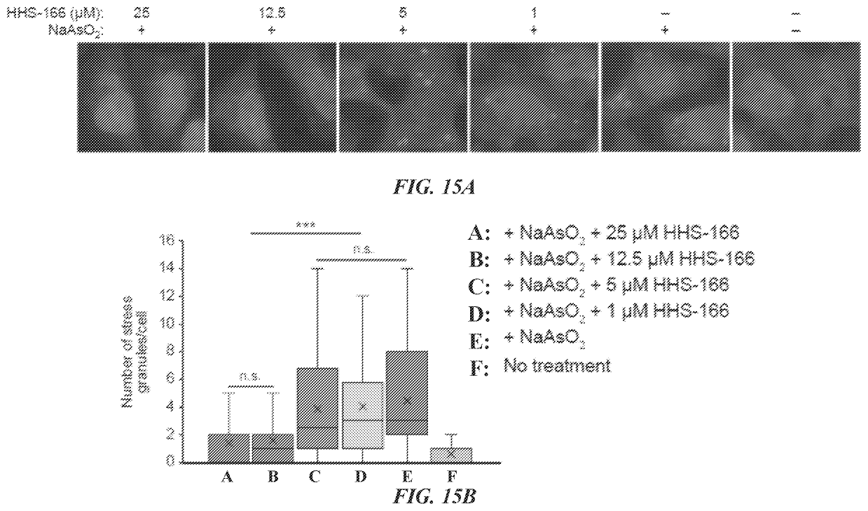

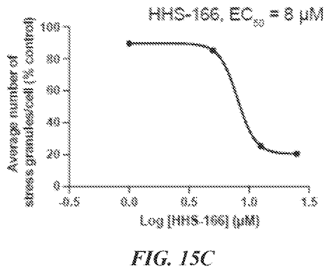

Our prioritization strategy identified key liganded PTM sites on SG proteins including G3BP1 (Y40, phosphorylation; Kim et al., 2022), HSPB1 (K123; acetylation, ubiquitination), and HNRPK (Y72, phosphorylation (Kim et al., 2017); see also FIGS. 5A and 13). The identification of G3BP1 was particularly interesting given its role as a nucleating protein for SGs and the identification of HHS-166 as a ligand for Y40 from our chemical proteomic studies (SR>2, FIGS. 5A, 5B, and 14). Importantly, the SG-inhibitory activity of HHS-166 was demonstrated to be dose dependent (EC₅₀=8 μM, LipE of ~3; FIG. 15). The Y40 site is located in the nuclear transport factor 2 domain (NTF2) of G3BP1, which has been shown to be important for G3BP1 dimerization, a key event for SG formation in vitro and in cells (FIG. 5C; Sanders et al., 2020; Yang et al., 2020). Interestingly, G3BP1 Y40 was not detected by global phosphoproteomic analysis of condensates (Sridharan et al., 2022) but was reported as a critical BTK-regulated phosphotyrosine for SG formation in response to viral infection (Kim et al., 2022).

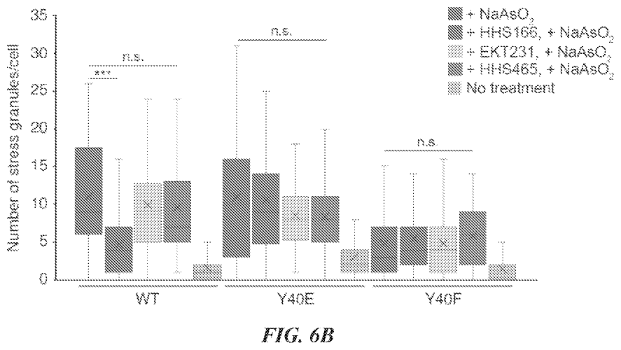

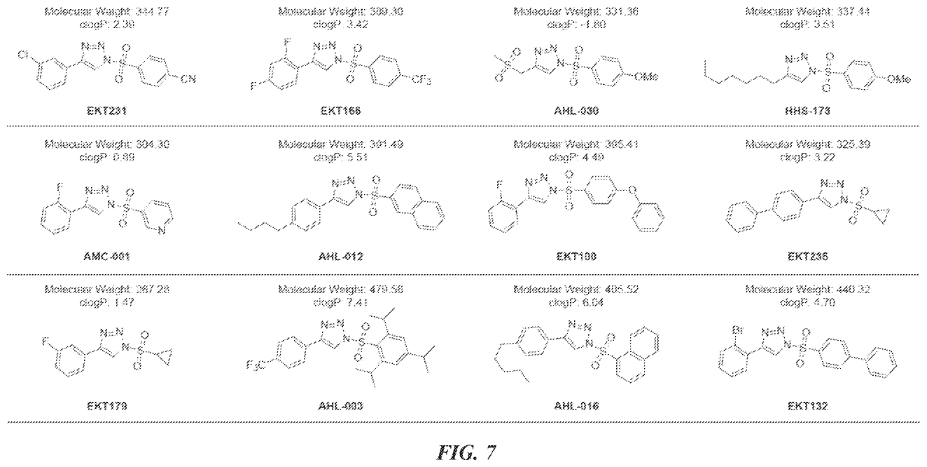

To determine whether the cell biological effects mediated by HHS-166 were Y40-dependent, we expressed recombinant G3BP1 WT or Y40 mutant in previously established G3BP1/2 double knockout U20S (G3BP KO) cells (Kedersha et al., 2016; Sanders et al., 2020) and evaluated the resulting effects on SG response to arsenite. We compared SG response of G3BP KO cells expressing a G3BP1 Y40 covalent binding- and phospho-deficient mutant (Y40F) with a phosphomimetic counterpart (Y40E). G3BP KO cells were previously shown to be deficient in arsenite-induced SG formation (Sanders et al., 2020). Expression of G3BP1 WT rescued this deficiency and restored cellular SG response to arsenite [1.6 vs 11 SGs/cell in (−)arsenite vs (+)arsenite, respectively; FIGS. 6A and 6B]. These G3BP1 WT-rescued cells responded to HHS-166 treatment, resulting in a statistically significant decrease in SGs (57% reduction in SGs/cell) that was not observed with pretreatment of a G3BP1 Y40- and SG-inactive compound (EKT231) or broad-reactive SuTEx probe (HHS-465, 9-13% reduction in SGs/cell for inactive compounds, respectively; FIG. 6B). Notably, expression of G3BP1 Y40F resulted in cells that were deficient in arsenite-induced SG formation and insensitive to SuTEx compound treatments. Stress-induced SG formation of G3BP1 Y40E-rescued cells was comparable to WT counterpart but insensitive to HHS-166 treatment, demonstrating the importance of Y40 for SuTEx ligandability and SG inhibition (FIGS. 6A and 6B).

In summary, our studies identify G3BP1 Y40 as a key regulatory site for arsenite-induced SG formation and show that covalent modification of this residue by the SuTEx ligand HHS-166 inhibits SG assembly.

### Discussion of the Examples

Aberrant condensate regulation is associated with a growing number of disease states (e.g., neurodegeneration, viral infection, cancer) and several therapeutic targets (TDP-43, FUS) are known to localize to these subcellular compartments (Mitrea et al., 2022). Targeting disease-relevant condensates offers unique opportunities for therapeutic discovery but has so far proven challenging due to the compositional diversity and dynamic nature of these evolutionarily conserved structures (Banani et al., 2017; Conti & Oppikofer, 2022). Here, we describe a covalent approach to discover condensate-modulating small molecules. The selection of SuTEx chemistry for our screening platform enabled access to ligandable Tyr/Lys residues, which are frequent in RNA-binding interfaces and can serve as sites for post-translational regulation, to perturb function of known RNP granule proteins as well as reveal new candidate targets proteome-wide.

We deployed a phenotypic screen for condensate-modulating small molecules by monitoring SG and PB formation in cells using established immunofluorescence markers (Kedersha & Anderson, 2007). Our previous chemical proteomic studies identified sulfonyl-triazoles (SuTEx) as a cell-active electrophile for covalent targeting of RNA-binding and protein-protein interaction domains (Hahm et al., 2020), which are known to facilitate high valency interactions for assembly of condensed RNP networks (Sanders et al., 2020). We pursued a fragment-based ligand discovery (FBLD) approach because of the ability to survey a larger fraction of chemical space with a smaller number of fragments (Erlanson et al., 2016). SuTEx was chosen for FBLD because LG diversification with binding groups permitted integration of the sulfone into fragment design as opposed to appending this electrophile to existing ligands (e.g., using SuFEx; Dong et al., 2014).

Our screen identified SuTEx compounds that inhibited SGs (EKT166, HHS-166), PBs (EKT132), and both types of RNP granules in stressed cells (AHL-003; FIGS. 2, 8, and 9). Unexpectedly, we also identified SuTEx ligands that enhanced stress-induced RNP granule levels in compound-treated cells. Pretreatment of cells with AMC-001 resulted in a statistically significant increase in PBs of glucose-deprived cells (>200% increase; FIGS. 2 and 8). These effects appeared specific for PBs as analogous pretreatments in arsenite-stressed cells resulted in negligible effects on the number of SGs per cell (FIG. 9).

We performed competitive LC-MS/MS ABPP studies to establish covalent binding profiles for active SuTEx compound hits. The outcome of these studies established, to the best of our knowledge, the first comprehensive map directly connecting RNP granule modulating activity with protein sites engaged by bioactive compounds in cells. In aggregate, we quantified >770 Tyr and Lys sites that are ligandable for developing covalent binders with RNP granule-modulating activity in cells. The proteomic reactivity of SuTEx electrophiles in cells (~3-9%) was comparable to hit rates previously reported for screening electrophile libraries (FIG. 3; Backus et al., 2016; Kuljanin et al., 2021).

Among the list of liganded RNP-granule proteins, we identified key RBPs (hnRNPA) and chaperone proteins (HSPB1) that have demonstrated roles in phase separation (Banani et al., 2017) or maintenance of condensates (Lu et al., 2022). The liganded sites mapped to functional domains of proteins that are reported sites for post-translational phosphorylation, ubiquitination, and sumoylation. By expanding the ligandable RNP granule proteome, the pharmacological tractability of individual proteins and sites within functional domains can be further explored to develop condensate-modulating compounds in future studies. Importantly, a subset of RNP granule proteins liganded by SuTEx fragments (~12%) were not detected in cell-based screens of large electrophile libraries of cysteine-reactive compounds (280+ members; Kuljanin et al., 2021). These findings highlight the need for Tyr/Lys-targeting chemistry for accessing RNP granule proteins that can be difficult to target with cysteine-reactive electrophiles.

Compared with previous Tyr-directed FBLD reports using 1,2,4-SuTEx compounds (Brulet et al., 2020), the current study identified a moderate preference for Lys compared with Tyr binding among the 1,2,3-SuTEx fragment hits (Y/K ratio of ~0.8, FIG. 11). This finding was important because it positions the largely underexplored 1,2,3-triazole LG as a feasible starting point for advancing SuTEx chemistry towards development of Lys-targeted ligands. The increased frequency of Lys among the liganded sites of SG/PB modulators was perhaps not surprising given that protein-RNA interfaces are abundant with Tyr and Lys residues and typically enriched for the latter to presumably mediate RNA phosphate recognition (Kruger et al., 2018). Thus, LG selection is an important criterion for guiding the future expansion of sulfone-based electrophile libraries to fully assess opportunities for chemical biology of RNP granules.

We demonstrated the utility of our integrated phenotypic screening and chemoproteomic approach through follow-up studies on compounds that affected dimerization domains of known RNP granule proteins. We identified AHL-030 as a unique hit compound because of its opposing activity to enhance SGs while modestly inhibiting PBs in stressed cells (FIG. 2). Competitive ABPP studies localized AHL-030 site of binding to Y475 in the YjeF_N domain of EDC3, which has been reported to function in self-dimerization (Ling et al., 2008) and mediate PB response to stress (FIG. 4; Ciancone et al., 2022). While additional studies are needed to understand AHL-030 mode of action, we demonstrated concentration-dependent binding to recombinant EDC3 that was specific for AHL-030 compared with other PB (EKT231) and PB/SG (AHL-003) modulators identified (FIGS. 4D-4F).

We provide evidence in support of site-specific activity for the SG-modulating compound HHS-166. This compound showed dose-dependent inhibition of arsenite-induced SG formation, and liganded G3BP1 Y40 in the NTF2 dimerization domain of this essential nucleating protein for SG regulation (FIGS. 5 and 15; Sanders et al., 2020; Yang et al., 2020). The SG modulating activity of HHS-166 was lost when covalent binding-deficient mutants (Y40E or Y40F) of G3BP1 were expressed in G3BP KO cells (FIG. 6). Further, the phosphodeficient Y40F G3BP1 mutant was impaired in arsenite-induced SG formation indicating that the Y40 phosphosite is a general regulatory site for both viral (Kim et al., 2022) and oxidative stress response of cells (FIG. 6).

There are a few limitations in our study that can be addressed in future studies. Our conclusions rely on counting RNP granules that are detectable by IF microscopy. There is some evidence that RNP granules exert cellular effects at sizes that do not provide a measurable optical phenotype. Additionally, the presence of microscopically visible RNP granules may depend on the IF protein marker utilized. In our studies, we selected G3BP1 and EDC4—both of which have been shown to be critical for RNP granule formation (Kedersha & Anderson, 2007)—as biomarkers of SG and PB structures, respectively. The inclusion of additional functional markers could further refine the evaluation of compositionally distinct RNP granules. We chose arsenite and glucose deprivation to induce RNP granules because these are widely adopted model systems (Kedersha & Anderson, 2007) but additional experimental conditions that capture disease-relevant condensate biology can also be tested (Banani et al., 2017). Our cellular screening conditions (25 μM SuTEx ligand treatment for 2 h) were chosen based on previous SuTEx compound screens in cells (Toroitich et al., 2021) that also matched conditions reported for cell-based screens of cysteine-reactive fragment electrophile libraries (Kuljanin et al., 2021). Additional screening efforts using SuTEx libraries, and fragment electrophiles in general, can identify appropriate compound concentrations and treatment times to address off-target activity and stability of reactive molecules in biological systems. Future work could also evaluate whether SuTEx chemoproteomics can be deployed for functional profiling of condensates found in different subcellular locations including the plasma membrane and nucleus (Banani et al., 2017).

Disclosed herein is thus a suite of electrophilic sulfonyl-triazole (SuTEx) compounds that modulate SG and PB levels in cells through covalent binding to central granule-forming RBPs. Active compounds from phenotypic screening were subjected to competitive activity-based protein profiling (ABPP) and quantitative proteomics to identify a collection of ~300 protein targets replete with ligandable tyrosine and lysine sites (>770 in aggregate). A substantial fraction of liganded proteins (~38%) were previously identified in proteomic analyses of RNP granules that included SG-nucleating (G3BP1) and PB-enhancing (EDC3) components. Importantly, we functionally validated G3BP1 Y40 as a ligandable site necessary for the SG-inhibitory activity of HHS-166 in oxidatively stressed cells. The presently disclosed findings support covalent binding at tyrosine and lysine residues as a global strategy for discovery of RNP granule modifiers.

In summary, presented herein is a systematic approach for discovering condensate-modulating covalent small molecules with defined protein interaction profiles to serve as chemical probes for investigating biomolecular condensate regulation and pharmacological tractability.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Aizer et al. The dynamics of mammalian P body transport, assembly, and disassembly in vivo. Mol Biol Cell 2008, 19 (10), 4154-4166. DOI: 10.1091/mbc.E08-05-0513.
Anderson et al. Stress granules, P-bodies and cancer. Biochim Biophys Acta 2015, 1849 (7), 861-870. DOI: 10.1016/j.bbagrm.2014.11.009.
Arimoto et al. Formation of stress granules inhibits apoptosis by suppressing stress-responsive MAPK pathways. Nat Cell Biol 2008, 10 (11), 1324-1332. DOI: 10.1038/ncb1791.
Ayache et al. P-body assembly requires DDX6 repression complexes rather than decay or Ataxin2/2L complexes. Mol Biol Cell 2015, 26 (14), 2579-2595. DOI: 10.1091/mbc.E15-03-0136.
Backus et al. Proteome-wide covalent ligand discovery in native biological systems. Nature 2016, 534 (7608), 570-574. DOI: 10.1038/nature18002.
Banani et al. Biomolecular condensates: organizers of cellular biochemistry. Nat Rev Mol Cell Biol 2017, 18 (5), 285-298. DOI: 10.1038/nrm.2017.7.
Baradaran-Heravi et al. Stress granule mediated protein aggregation and underlying gene defects in the FTD-ALS spectrum. Neurobiol Dis 2020, 134, 104639. DOI: 10.1016/j.nbd.2019.104639.
Bloch et al. GW/P-bodies and autoimmune disease. Adv Exp Med Biol. 2013:768:61-70.
Bordeleau et al. (2005) Stimulation of mammalian translation initiation factor eIF4A activity by a small molecule inhibitor of eukaryotic translation. Proc Natl Acad Sci USA 102:10460-10465.
Bordeleau et al. Functional characterization of IRESes by an inhibitor of the RNA helicase eIF4A. Nat Chem Biol 2006, 2 (4), 213-220. DOI: 10.1038/nchembio776.
Bordeleau et al. Stimulation of mammalian translation initiation factor eIF4A activity by a small molecule inhibitor of eukaryotic translation. Proc Natl Acad Sci USA 2005, 102 (30), 10460-10465. DOI: 10.1073/pnas.0504249102.
Brengues et al. Movement of eukaryotic mRNAs between polysomes and cytoplasmic processing bodies. Science 2005, 310 (5747), 486-489. DOI: 10.1126/science.1115791.
Brulet et al. Liganding Functional Tyrosine Sites on Proteins Using Sulfur-Triazole Exchange Chemistry. J Am Chem Soc 2020, 142 (18), 8270-8280. DOI: 10.1021/jacs.0c00648.
Buchan. mRNP granules: Assembly, function, and connections with disease. RNA Biol. 2014; 11(8):1019-1030.
Cai et al. G3BP1 Inhibition Alleviates Intracellular Nucleic Acid-Induced Autoimmune Responses. J Immunol 2021, 206 (10), 2453-2467. DOI: 10.4049/jimmunol.2001111.
Ciancone et al. Global profiling identifies a stress-responsive tyrosine site on EDC3 regulating biomolecular condensate formation. Cell Chem Biol 2022, 29 (12), 1709-1720 e1707. DOI: 10.1016/j.chembiol.2022.11.008.
Collier & Schlesinger. The dynamic state of heat shock proteins in chicken embryo fibroblasts. J Cell Biol 1986, 103 (4), 1495-1507. DOI: 10.1083/jcb.103.4.1495.
Colombrita et al. TDP-43 is recruited to stress granules in conditions of oxidative insult. J Neurochem 2009, 111 (4), 1051-1061. DOI: 10.1111/j.1471-4159.2009.06383.x.
Conti & Oppikofer. Biomolecular condensates: new opportunities for drug discovery and RNA therapeutics. Trends Pharmacol Sci 2022, 43 (10), 820-837. DOI: 10.1016/j.tips.2022.07.001.
Cougot et al. Cytoplasmic foci are sites of mRNA decay in human cells. J Cell Biol 2004, 165 (1), 31-40. DOI: 10.1083/jcb.200309008.
Cui et al. Diverse CMT2 neuropathies are linked to aberrant G3BP interactions in stress granules. Cell 2023. DOI: 10.1016/j.cell.2022.12.046.
Decker & Parker. P-bodies and stress granules: possible roles in the control of translation and mRNA degradation. Cold Spring Harb Perspect Biol 2012, 4 (9), a012286. DOI: 10.1101/cshperspect.a012286.
Dong et al. Sulfur(VI) fluoride exchange (SuFEx): another good reaction for click chemistry. Angew Chem Int Ed Engl 2014, 53 (36), 9430-9448. DOI: 10.1002/anie.201309399.
Erlanson et al. Twenty years on: the impact of fragments on drug discovery. Nat Rev Drug Discov 2016, 15 (9), 605-619. DOI: 10.1038/nrd.2016.109.
Fang et al. Small-Molecule Modulation of TDP-43 Recruitment to Stress Granules Prevents Persistent TDP-43 Accumulation in ALS/FTD. Neuron 2019, 103 (5), 802-819 e811. DOI: 10.1016/j.neuron.2019.05.048.
Fournier et al. The chemotherapeutic agent bortezomib induces the formation of stress granules. Cancer Cell Int 2010, 10, 12. DOI: 10.1186/1475-2867-10-12.
Franks & Lykke-Andersen (2007) TTP and BRF proteins nucleate processing body formation to silence mRNAs with AU-rich elements. Genes Dev 21, 719-735.
Fujimura et al. Selenite targets eIF4E-binding protein-1 to inhibit translation initiation and induce the assembly of non-canonical stress granules. Nucleic Acids Res 2012, 40 (16), 8099-8110. DOI: 10.1093/nar/gks566.
Garcia et al. (2006) Impact of protein kinase PKR in cell biology: from antiviral to antiproliferative action. Microbiol Mol Biol Rev 70:1032-1060.
Garcia-Maurino et al. (2017) RNA Binding Protein Regulation and Cross-Talk in the Control of AU-rich mRNA Fate. Front Mol Biosci 4:71.
Gennaro (ed.) (1985) Remington's Pharmaceutical Sciences, Mack Publishing Co., Easton, Pennsylvania, United States of America.
Gennaro (ed.) (1990) Remington's Pharmaceutical Sciences, 18th ed., Mack Pub. Co., Easton, Pennsylvania, United States of America,
Gennaro (ed.) (2003) Remington: The Science and Practice of Pharmacy, 20th edition Lippincott, Williams & Wilkins, Philadelphia, Pennsylvania, United States of America.
Gordon et al. A SARS-CoV-2-Human Protein-Protein Interaction Map Reveals Drug Targets and Potential Drug-Repurposing. bioRxiv 2020. DOI: 10.1101/2020.03.22.002386.
Grams & Hsu. Reactive chemistry for covalent probe and therapeutic development. Trends Pharmacol Sci 2022, 43 (3), 249-262. DOI: 10.1016/j.tips.2021.12.002.
Guillen-Boixet et al. RNA-Induced Conformational Switching and Clustering of G3BP Drive Stress Granule Assembly by Condensation. Cell 2020, 181 (2), 346-361 e317. DOI: 10.1016/j.cell.2020.03.049.
Hahm et al. Global targeting of functional tyrosines using sulfur-triazole exchange chemistry. Nat Chem Biol 2020, 16 (2), 150-159. DOI: 10.1038/s41589-019-0404-5.
Hallacli et al. The Parkinson's disease protein alpha-synuclein is a modulator of processing bodies and mRNA stability. Cell 2022, 185 (12), 2035-2056 e2033. DOI: 10.1016/j.cell.2022.05.008.
Hans et al. Multiple distinct pathways lead to hyperubiquitylated insoluble TDP-43 protein independent of its translocation into stress granules. J Biol Chem 2020, 295 (3), 673-689. DOI: 10.1074/jbc.RA119.010617.
Hentze et al. A brave new world of RNA-binding proteins. Nat Rev Mol Cell Biol 2018, 19 (5), 327-341. DOI: 10.1038/nrm.2017.130.
Hofweber & Dormann. Friend or foe-Post-translational modifications as regulators of phase separation and RNP granule dynamics. J Biol Chem 2019, 294 (18), 7137-7150. DOI: 10.1074/jbc.TM118.001189.
Huang et al. Chemoproteomic profiling of kinases in live cells using electrophilic sulfonyl triazole probes. Chemical Science 2021, 12 (9), 3295-3307, 10.1039/D0SC06623K. DOI: 10.1039/d0sc06623k.
Hubstenberger et al. P-Body Purification Reveals the Condensation of Repressed mRNA Regulons. Mol Cell 2017, 68 (1), 144-157 e145. DOI: 10.1016/j.molcel.2017.09.003.
Ivanov et al. Stress Granules and Processing Bodies in Translational Control. Cold Spring Harb Perspect Biol 2019, 11 (5). DOI: 10.1101/cshperspect.a032813.
Jones & Kelly. Structure-based design and analysis of SuFEx chemical probes. RSC Med Chem 2020, 11 (1), 10-17. DOI: 10.1039/c9md00542k.
Kaehler et al. 5-Fluorouracil affects assembly of stress granules based on RNA incorporation. Nucleic Acids Res 2014, 42 (10), 6436-6447. DOI: 10.1093/nar/gku264.
Kedersha & Anderson. Mammalian Stress Granules and Processing Bodies. In Translation Initiation: Cell Biology, High-Throughput Methods, and Chemical-Based Approaches, In Methods in Enzymology, 2007; pp 61-81.
Kedersha et al. G3BP-Caprin1-USP10 complexes mediate stress granule condensation and associate with 40S subunits. J Cell Biol 2016, 212 (7), 845-860. DOI: 10.1083/jcb.201508028.
Kim et al. Bruton's Tyrosine Kinase phosphorylates scaffolding and RNA-binding protein G3BP1 to induce stress granule aggregation during host sensing of foreign ribonucleic acids. J Biol Chem 2022, 102231. DOI: 10.1016/j.jbc.2022.102231.
Kim et al. Heterogeneous nuclear ribonucleoprotein K inhibits heat shock-induced transcriptional activity of heat shock factor 1. J Biol Chem 2017, 292 (31), 12801-12812. DOI: 10.1074/jbc.M117.774992.
Kruger et al. Protein-RNA interactions: structural characteristics and hotspot amino acids. RNA 2018, 24 (11), 1457-1465. DOI: 10.1261/ma.066464.118.
Kuljanin et al. Reimagining high-throughput profiling of reactive cysteines for cell-based screening of large electrophile libraries. Nat Biotechnol 2021, 39 (5), 630-641. DOI: 10.1038/s41587-020-00778-3.
Kwon et al. The deacetylase HDAC6 is a novel critical component of stress granules involved in the stress response. Genes Dev 2007, 21 (24), 3381-3394. DOI: 10.1101/gad.461107.
Lavalee et al. Cancer cell adaptability: turning ribonucleoprotein granules into targets. Trends Cancer 2021, 7 (10), 902-915. DOI: 10.1016/j.trecan.2021.05.006.
Leeson & Springthorpe. The influence of drug-like concepts on decision-making in medicinal chemistry. Nat Rev Drug Discov 2007, 6 (11), 881-890. DOI: 10.1038/nrd2445.
Ling et al. Crystal structure of human Edc3 and its functional implications. Mol Cell Biol 2008, 28 (19), 5965-5976. DOI: 10.1128/MCB.00761-08.
Lloyd. Regulation of stress granules and P-bodies during RNA virus infection. Wiley Interdiscip Rev RNA. 2013 May-June; 4(3):317-331.
Low et al. Inhibition of eukaryotic translation initiation by the marine natural product pateamine A. Mol Cell 2005, 20 (5), 709-722. DOI: 10.1016/j.molcel.2005.10.008.
Lu et al. Heat-shock chaperone HSPB1 regulates cytoplasmic TDP-43 phase separation and liquid-to-gel transition. Nat Cell Biol 2022, 24 (9), 1378-1393. DOI: 10.1038/s41556-022-00988-8.
Luo et al. P-Bodies: Composition, Properties, and Functions. Biochemistry 2018, 57 (17), 2424-2431. DOI: 10.1021/acs.biochem.7b01162.
Mackenzie et al. TIA1 Mutations in Amyotrophic Lateral Sclerosis and Frontotemporal Dementia Promote Phase Separation and Alter Stress Granule Dynamics. Neuron 2017, 95 (4), 808-816 e809. DOI: 10.1016/j.neuron.2017.07.025.
Markmiller et al. Context-Dependent and Disease-Specific Diversity in Protein Interactions within Stress Granules. Cell 2018, 172 (3), 590-604 e513. DOI: 10.1016/j.cell.2017.12.032.
Marmor-Kollet et al. Spatiotemporal Proteomic Analysis of Stress Granule Disassembly Using APEX Reveals Regulation by SUMOylation and Links to ALS Pathogenesis. Mol Cell 2020, 80 (5), 876-891 e876. DOI: 10.1016/j.molcel.2020.10.032.
Mi et al. Large-scale gene function analysis with the PANTHER classification system. Nat Protoc 2013, 8 (8), 1551-1566. DOI: 10.1038/nprot.2013.092.
Mitrea et al. Modulating biomolecular condensates: a novel approach to drug discovery. Nat Rev Drug Discov 2022, 21 (11), 841-862. DOI: 10.1038/s41573-022-00505-4.
Monahan et al. Phosphorylation of the FUS low-complexity domain disrupts phase separation, aggregation, and toxicity. EMBO J 2017, 36 (20), 2951-2967. DOI: 10.15252/embj.201696394.
Nielsen et al. Cytoplasmic trafficking of IGF-II mRNA-binding protein by conserved KH domains. J Cell Sci 2002, 115 (Pt 10), 2087-2097. DOI: 10.1242/jcs.115.10.2087.
Nover et al. Cytoplasmic heat shock granules are formed from precursor particles and are associated with a specific set of mRNAs. Mol Cell Biol 1989, 9 (3), 1298-1308. DOI: 10.1128/mcb.9.3.1298-1308.1989.
Patel et al. Principles and functions of condensate modifying drugs. Frontiers in Molecular Biosciences 2022, 9, Review. DOI: 10.3389/fmolb.2022.1007744.
Pattabiraman et al. Vimentin protects differentiating stem cells from stress. Sci Rep 2020, 10 (1), 19525. DOI: 10.1038/s41598-020-76076-4.
Protter & Parker. Principles and Properties of Stress Granules. Trends Cell Biol 2016, 26 (9), 668-679. DOI: 10.1016/j.tcb.2016.05.004.
Reineke & Lloyd. The stress granule protein G3BP1 recruits protein kinase R to promote multiple innate immune antiviral responses. J Virol 2015, 89 (5), 2575-2589. DOI: 10.1128/JVI.02791-14.
Riggs et al. Mammalian stress granules and P bodies at a glance. J Cell Sci 2020, 133 (16). DOI: 10.1242/jcs.242487.
Sanders et al. Competing Protein-RNA Interaction Networks Control Multiphase Intracellular Organization. Cell 2020, 181 (2), 306-324 e328. DOI: 10.1016/j.cell.2020.03.050.
Sheils et al. TCRD and Pharos 2021: mining the human proteome for disease biology. Nucleic Acids Res 2021, 49 (D1), D1334-D1346. DOI: 10.1093/nar/gkaa993.
Sheth & Parker. Decapping and decay of messenger RNA occur in cytoplasmic processing bodies. Science 2003, 300 (5620), 805-808. DOI: 10.1126/science.1082320.
Shin & Brangwynne. Liquid phase condensation in cell physiology and disease. Science 2017, 357 (6357). DOI: 10.1126/science.aaf4382.
Sreedharan et al. TDP-43 mutations in familial and sporadic amyotrophic lateral sclerosis. Science 2008, 319 (5870), 1668-1672. DOI: 10.1126/science.1154584.
Sridharan et al. Systematic discovery of biomolecular condensate-specific protein phosphorylation. Nat Chem Biol 2022, 18 (10), 1104-1114. DOI: 10.1038/s41589-022-01062-y.
Standart & Weil. P-Bodies: Cytosolic Droplets for Coordinated mRNA Storage. Trends Genet 2018, 34 (8), 612-626. DOI: 10.1016/j.tig.2018.05.005.
Stoecklin & Kedersha. Relationship of GW/P-bodies with stress granules. Adv Exp Med Biol 2013, 768, 197-211. DOI: 10.1007/978-1-4614-5107-5_12.
Takahashi et al. (2011) RhoA activation participates in rearrangement of processing bodies and release of nucleated AU-rich mRNAs. Nucleic Acids Res 39, 3446-3457.
Toivola et al. Intermediate filaments take the heat as stress proteins. Trends Cell Biol 2010, 20 (2), 79-91. DOI: 10.1016/j.tcb.2009.11.004.
Toroitich et al. Discovery of a Cell-Active SuTEx Ligand of Prostaglandin Reductase 2. Chembiochem 2021, 22 (12), 2134-2139. DOI: 10.1002/cbic.202000879.
Tsang et al. Phosphoregulated FMRP phase separation models activity-dependent translation through bidirectional control of mRNA granule formation. Proc Natl Acad Sci USA 2019, 116 (10), 4218-4227. DOI: 10.1073/pnas.1814385116.
Vanderweyde et al. Role of stress granules and RNA-binding proteins in neurodegeneration: a mini-review. Gerontology. 2013; 59:524-533. [PubMed: 24008580]
Yang et al. G3BP1 Is a Tunable Switch that Triggers Phase Separation to Assemble Stress Granules. Cell 2020, 181 (2), 325-345 e328. DOI: 10.1016/j.cell.2020.03.046.
Youn et al. Properties of Stress Granule and P-Body Proteomes. Mol Cell 2019, 76 (2), 286-294. DOI: 10.1016/j.molcel.2019.09.014.
Zheng et al. Previously uncharacterized interactions between the folded and intrinsically disordered domains impart asymmetric effects on UBQLN2 phase separation. Protein Sci 2021, 30 (7), 1467-1481. DOI: 10.1002/pro.4128.

It will be understood that various details of the presently disclosed subject matter can be changed without departing from the scope of the presently disclosed subject matter. Furthermore, the foregoing description is for the purpose of illustration only, and not for the purpose of limitation.

1. A method for modulating the formation of ribonucleoprotein granules (RNPGs), the method comprising contacting a sample comprising a protein with a sulfonyl triazole compound, thereby covalently modifying the protein.

2. The method of claim 1, wherein the RNPG is a stress granule (SG) or a processing body (PB) and/or wherein the sulfonyl triazole compound is capable of covalently modifying an RNA granule induction-sensitive lysine and tyrosine site (RISKY) in the protein.

3. The method of claim 1, wherein the sulfonyl triazole compound has a structure of Formula (I): 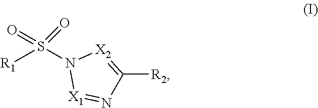
wherein:
X₁ and X₂ are each CH or N, subject to the proviso that when X₁ is CH, X₂ is N and when X₁ is N, X₂ is CH;
R₁ is selected from alkyl, cycloalkyl, aralkyl, aryl, and substituted aryl; and
R₂ is selected from alkyl, substituted alkyl, aralkyl, substituted aralkyl, aryl, and substituted aryl.

4. The method of claim 3, wherein X₁ is N and X₂ is CH.

5. The method of claim 3, wherein R₁ is selected from the group consisting of isopropyl, cyclopropyl, naphthyl, pyridyl, phenyl, and substituted phenyl.

6. The method of claim 5, wherein R₁ is substituted phenyl, wherein said substituted phenyl is phenyl substituted with one or more substituents selected from the group consisting of cyano, halo, alkyl, alkoxy, perfluoroalkyl, perfluoroalkoxy, phenyl, and —NH(═O)-alkyl, optionally wherein substituted phenyl is phenyl substituted with one or more substituents selected from the group consisting of cyano, bromo, fluoro, methyl, isopropyl, methoxy, trifluoromethyl, trifluoromethoxy, phenyl, and —NH(═O)-methyl.

7. The method of claim 3, wherein R₂ is selected from the group consisting of alkyl, sulfonyl-substituted alkyl, cycloalkyl, phenyl, pyridyl, and substituted phenyl.

8. The method of claim 7, wherein R₂ is substituted phenyl, wherein said substituted phenyl is phenyl substituted with one or more substituents selected from the group consisting of halo, alkyl, perfluoroalkyl, alkoxy, and phenyl, optionally, fluoro, bromo, chloro, butyl, pentyl, trifluoromethyl, methoxy, and phenyl.

9. The method of claim 7, wherein R₂ is selected from the group consisting of heptyl, hexyl, cyclohexyl, —CH₂—S(═O)₂—CH₃, pyridyl, and phenyl.

10. The method of claim 1, wherein the modulating inhibits the formation of PB and/or SG, optionally wherein the compound is selected from the group consisting of:

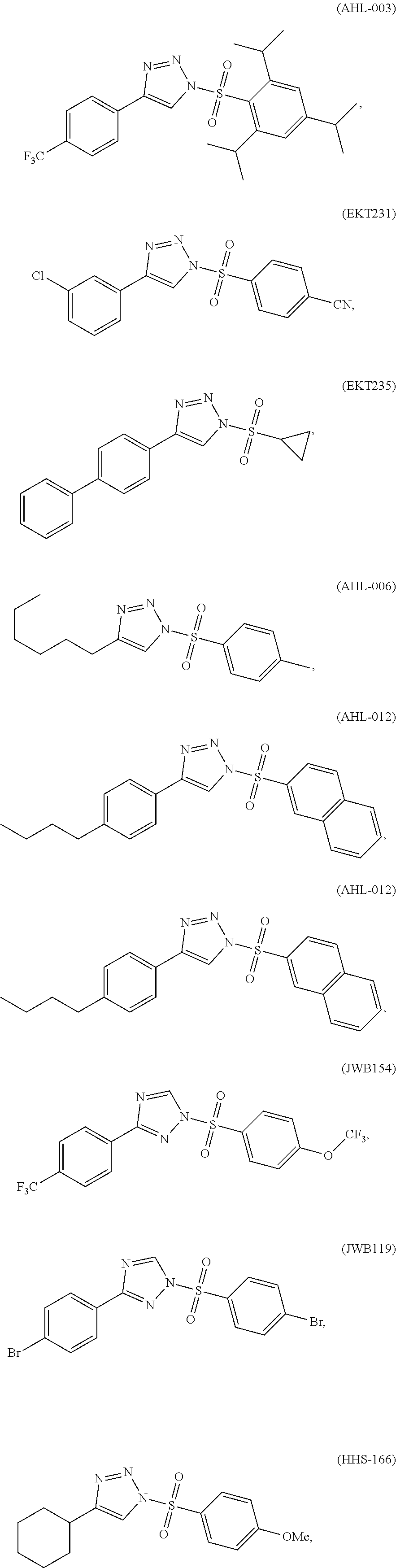

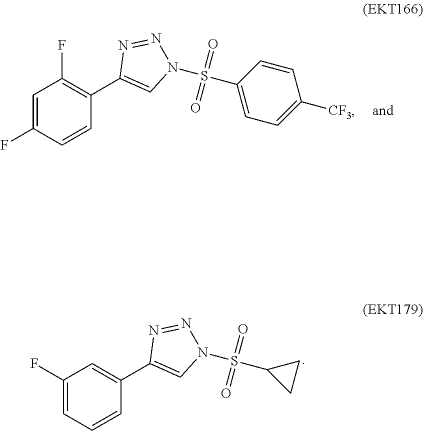 [SMILES: O=S(=O)(c1ccc(C(F)(F)F)cc1)n1cc(-c2ccc(F)cc2F)nn1 ; "and" ; O=S(=O)(C1CC1)n1cc(-c2cccc(F)c2)nn1]

11. The method of claim 1, wherein the modulation induces the formation of PB and/or SG, optionally wherein the compound is selected from the group consisting of:

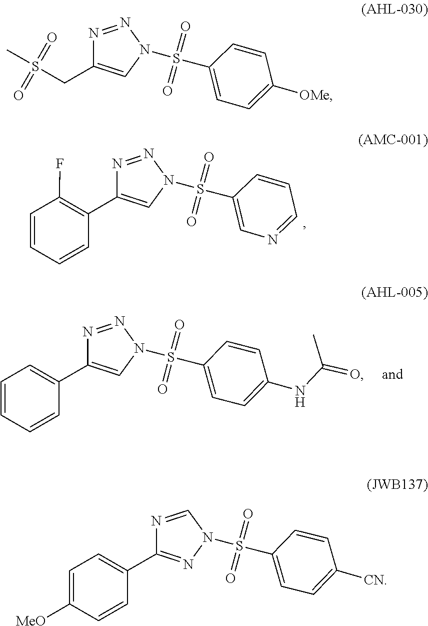 [SMILES: COc1ccc(S(=O)(=O)n2cc(CS(C)(=O)=O)nn2)cc1 ; O=S(=O)(c1cccnc1)n1cc(-c2ccccc2F)nn1 ; CC(=O)Nc1ccc(S(=O)(=O)n2cc(-c3ccccc3)nn2)cc1 ; "and" ; COc1ccc(-c2ncn(S(=O)(=O)c3ccc(C#N)cc3)n2)cc1]

12. The method of claim 1, wherein the protein is anti-RAS GTPase-activating protein-binding protein 1 (G3BP1), Parkinson diseases protein 1 (PARK7), or enhancer of mRNA-decapping protein 3 (EDC3).

13. The method of claim 1, wherein modulating the formation of RNPGs comprises inhibiting the multimerization of G3BP1, optionally wherein the method comprises contacting a sample comprising G3BP1 with

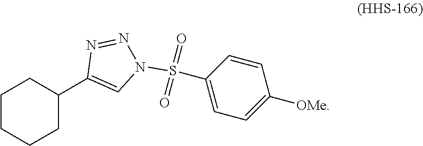 [SMILES: COc1ccc(S(=O)(=O)n2cc(C3CCCCC3)nn2)cc1]

14. The method of claim 1, wherein modulating the formation of RNPGs comprises inhibiting the multimerization of PARK7, optionally wherein the method comprising contacting a sample comprising the PARK7 with

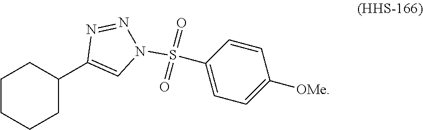 [SMILES: COc1ccc(S(=O)(=O)n2cc(C3CCCCC3)nn2)cc1]

15. The method of claim 1, wherein modulating the formation of RNPGs comprises inhibiting the formation of PGs or increasing the formation of SGs by modulating the multimerization of EDC3, optionally wherein the method comprising contacting a sample comprising the EDC3 with

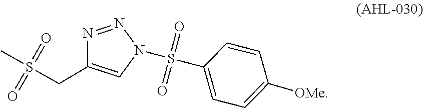 [SMILES: COc1ccc(S(=O)(=O)n2cc(CS(C)(=O)=O)nn2)cc1]

16. A method for treating a symptom of a disease or infection in a subject in need thereof, wherein the method comprises administering to the subject an effective amount of a sulfonyl triazole compound of Formula (I): 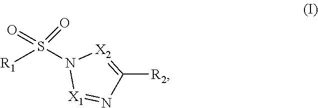
wherein:
X₁ and X₂ are each CH or N, subject to the proviso that when X₁ is CH, X₂ is N and when X₁ is N, X₂ is CH;
R₁ is selected from alkyl, cycloalkyl, aralkyl, aryl, and substituted aryl; and
R₂ is selected from alkyl, substituted alkyl, aralkyl, substituted aralkyl, aryl, and substituted aryl, wherein said administrating modulates the formation of pathological ribonucleoprotein granules (RNPGs).

17. The method of claim 16, wherein the disease or infection is selected from a viral infection, cancer, and a neurological disease, optionally frontotemporal dementia or Parkinson's disease.

18. A sulfonyl triazole compound, wherein said sulfonyl triazole compound is selected from the group consisting of:

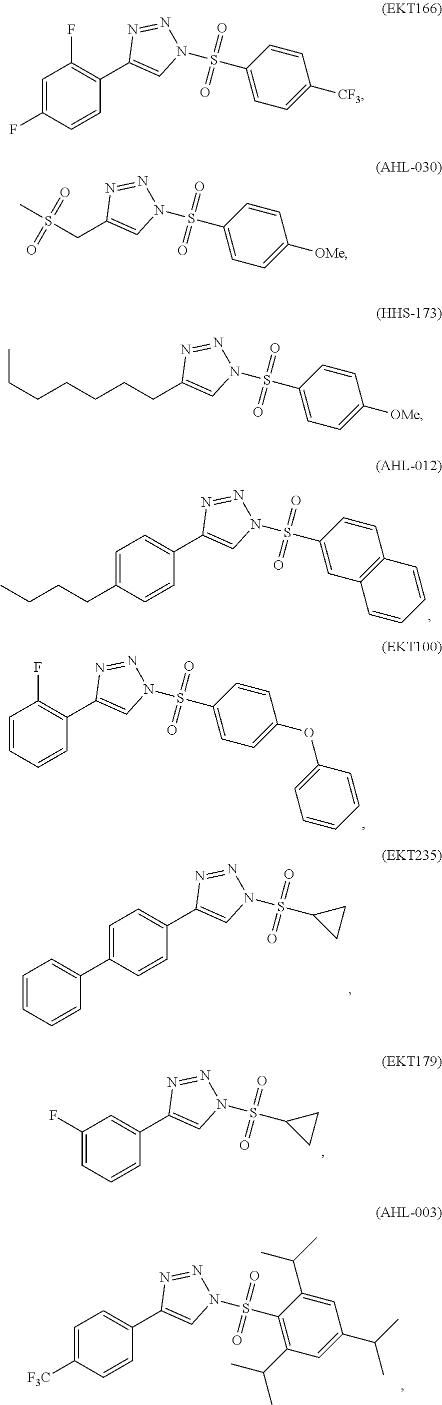

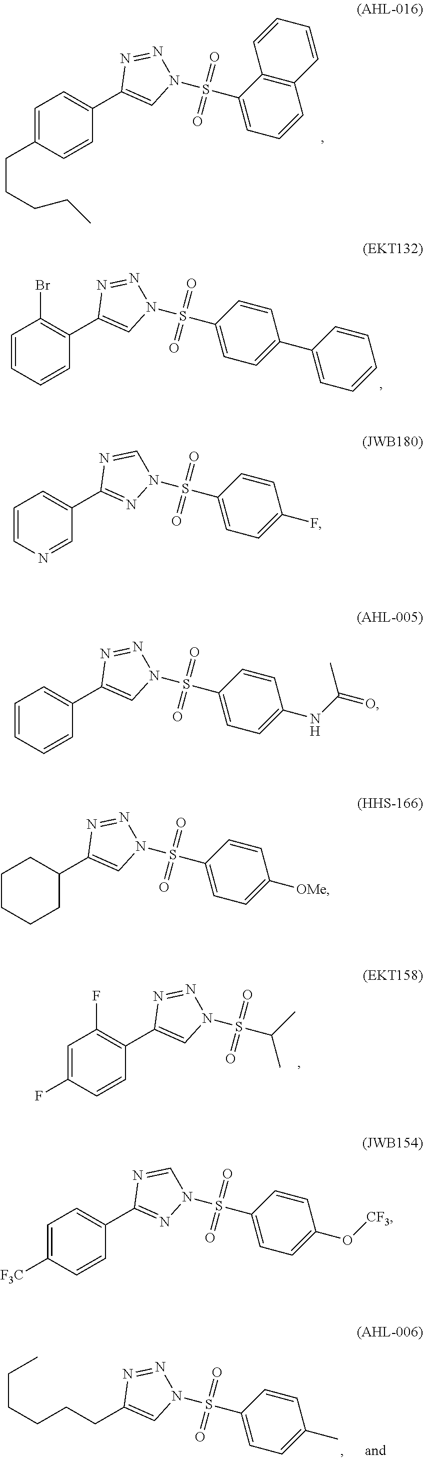

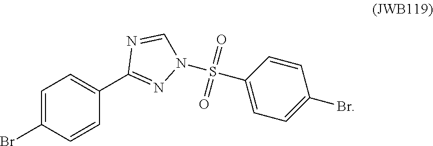 [SMILES: O=S(=O)(c1ccc(Br)cc1)n1cnc(-c2ccc(Br)cc2)n1]

19. The sulfonyl triazole compound of claim 15, wherein said compound is selected from:

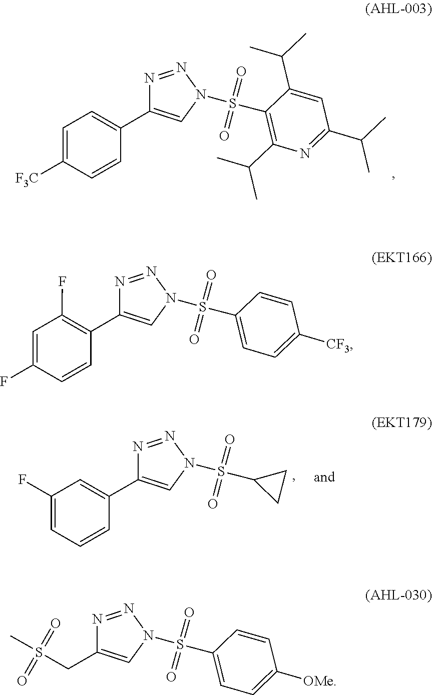

20. A pharmaceutical composition comprising a compound of claim 18 and a pharmaceutical carrier.

21. A composition for use in treating a symptom associated with a viral infection, cancer, or a neurological disease, wherein the composition comprises a compound of Formula (I): 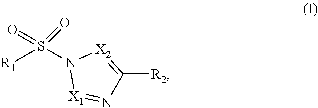
wherein:
X₁ and X₂ are each CH or N, subject to the proviso that when X₁ is CH, X₂ is N and when X₁ is N, X₂ is CH;
R₁ is selected from alkyl, cycloalkyl, aralkyl, aryl, and substituted aryl; and
R₂ is selected from alkyl, substituted alkyl, aralkyl, substituted aralkyl, aryl, and substituted aryl, wherein said administrating modulates the formation of pathological ribonucleoprotein granules (RNPGs).


SEQUENCE LISTING (3 sequences)

SEQ ID NO: 1 - protein, 27 aa, Homo sapiens
IYLCDIGIPQQVFQEVGINYHSPFGCK

SEQ ID NO: 2 - protein, 23 aa, Homo sapiens
NSSYVHGGLDSNGKPADAVYGQK

SEQ ID NO: 3 - protein, 22 aa, Homo sapiens
DGVVEITGKHEERQDEHGYISR